(19) United States
(12) Patent Application Publication
(10) Pub. No.: US 2024/0173686 A1
(43) Pub. Date: May 30, 2024

(54) LIPID NANOPARTICLE (LNP) ENCAPSULATION OF MRNA PRODUCTS

(71) Applicant: Nature's Toolbox, Inc., Rio Rancho, NM (US)

(72) Inventor: Alexander Koglin, Santa Fe, NM (US)

(21) Appl. No.: 18/237,251

(22) Filed: Aug. 23, 2023

Related U.S. Application Data
(63) Continuation-in-part of application No. PCT/US22/17531, filed on Feb. 23, 2022.
(60) Provisional application No. 63/152,460, filed on Feb. 23, 2021.

(51) Int. Cl. B01J 13/10; A61K 9/51; B01L 3/00

(52) U.S. Cl. CPC B01J 13/10; B01L 3/502761; A61K 9/5123; B01L 2200/0647; B01L 2300/0816; B01L 2300/088

(57) ABSTRACT

The invention includes a novel microfluidic mixing chip configured for the production of lipid nano-particles (LNPs) and in particular LNPs encapsulating oligonucleotides, such as mRNA that may be used in various therapeutic applications such as vaccines and the like.

### CROSS-REFERENCE TO RELATED APPLICATIONS

This International PCT Application claims the benefit of and priority to U.S. Provisional Application No. 63/152,460 filed Feb. 23, 2022. The entire specification and figures of the above-referenced application is hereby incorporated, in their entirety by reference.

### TECHNICAL FIELD

The invention relates to novel systems, methods and apparatus for the improved encapsulation of small molecules, and in particular the encapsulation of Ribonucleic Nucleic Acid (RNA) products within a lipid nanoparticle (LNP).

### BACKGROUND

The production and delivery of therapeutic nucleic acids has received increased interest in light of the world-wide COVID pandemic and subsequent production of first generation mRNA-based vaccines that elicited a strong immunity response against the disease. Despite this initial progress, there are still technical shortcomings that limit mRNA vaccine's widespread adoptions. For example, there remains a need for more effective delivery of RNA vaccines, and other nucleic acid-based therapeutics, to appropriate sites within a cell or organism in order to realize this potential. However, two main problems currently limit the effectiveness of nucleic acid-based therapeutics.

First, free RNAs are susceptible to nuclease digestion in plasma. Second, free RNAs have limited ability to gain access to the intracellular compartment where the relevant translation machinery resides. To overcome these problems, Lipid nanoparticles (LNP) formed from cationic lipids with other lipid components, such as neutral lipids, cholesterol, PEG, PEGylated lipids, and oligonucleotides have been used to block degradation of the RNAs in plasma and facilitate the cellular uptake of the oligonucleotides. However, the use of LNPs also presents its own challenges. For example, the production and encapsulation of consistent quantities of nucleic acid therapeutics in uniformly sized LNPs has proven difficult. In addition, such LNPs must be kept in many cases in extremely cold temperatures, such as −80° C. or below, rendering them almost useless in locations that lack such expansive refrigeration capacity and infrastructure.

As can be seen, there exists a need for a cost-effective and efficient LNP production and nucleic acid packing system that addresses the concerns outlined above.

### SUMMARY OF THE INVENTION

One aspect of the invention includes a novel microfluidic mixing chip configured for the production of LNPs, and in particular LNPs encapsulating oligonucleotides, such as mRNA that may be used in various therapeutic applications such as vaccines and the like.

Another aspect of the invention includes a novel microfluidic mixing chip configured to have one or more channel features that increases the efficiency of oligonucleotide encapsulation, as well as promote the production of LNPs having a smaller and more consistently spherical particle size, or diameter than traditional LNP production methods.

Another aspect of the invention includes a lipid solution for the production of LNPs using, in a preferred aspect the novel microfluidic mixing chip of the invention, wherein this lipid solution of the invention increases the efficiency of oligonucleotide encapsulation, as well as promote the production of LNPs having a smaller and more consistently spherical particle size, or diameter than traditional LNP production methods.

Another aspect of the invention includes a lipid solution for the production of LNPs using, in a preferred aspect the novel microfluidic mixing chip of the invention, wherein the LNPs produced by the methods and apparatus of the invention can be lyophilized and reconstituted while demonstrating enhanced retention of oligonucleotide.

Another embodiment aspect of the invention includes LNPs produced by the methods and apparatus of the invention, that may further be used as a pharmaceutical composition, which may preferably be a vaccine.

Another aspect of the invention include a microfluidic mixing chip for the production of lipid nano-particles (LNPs) having: at least one lipid insertion channel; one or more buffer channels; an inlet junction; and a mixing channel fluid, wherein said mixing channel optionally includes one or more of: a meander channel, and one or more hairpin turns. In a preferred aspect, the microfluidic mixing chip of the invention may include one or more of the aforementioned elements and may further be selected from the group consisting of:

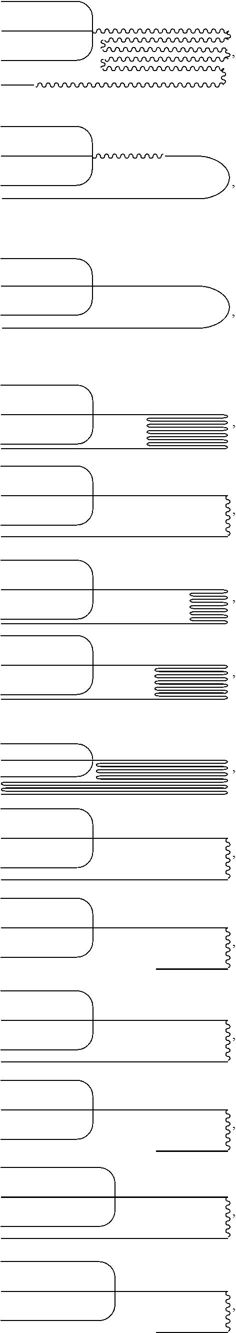

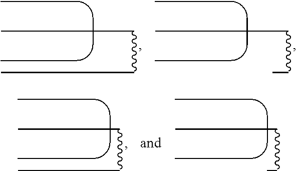

Other aspects and features of the present invention will become apparent to those ordinarily skilled in the art upon review of the following descriptions of specific embodiments of the invention in conjunction with the accompanying figures.

### BRIEF DESCRIPTION OF THE FIGURES

The above and other aspects, features, and advantages of the present disclosure will be better understood from the following detailed descriptions taken in conjunction with the accompanying figures, all of which are given by way of illustration only, and are not limiting the presently disclosed embodiments, in which:

FIGS. 1A-1B. Top: (FIG. 1A) exemplary droplet microfluidic mixing chip being in this embodiment approximately 1 inch×3 inches. Bottom: (FIG. 1B) microfluidic mixing chip mounted in header and ready to undergo initial flow test.

FIG. 2. Results of real time flow tests using ethanolic tartrazine (λ_{EtOH}) and aqueous methylene blue (λ_{aq}).

FIG. 3. Schematic representation of droplet microfluidic chip used for lipid nanoparticle formation. CA/CC: 112 μL/min. CB: 75 μL/min. OC: 299 μL/min.

FIGS. 4A-4B. (FIG. 4A, FIG. 4B) Dynamic light scattering of lipid nanoparticles obtained as a function of time.

FIG. 5A-5C. Dynamic light scattering of lipid nanoparticles obtained as a function of time using new microchip having twice the channel depth. (FIG. 5A) throughput of 300 μL/min, 25 vol. % ethanolic lipid mixture. (FIG. 5B) throughput of 600 μL/min, 25 vol. % ethanolic lipid mixture. (FIG. 5C) throughput of 300 μL/min, 10 vol. % ethanolic lipid mixture.

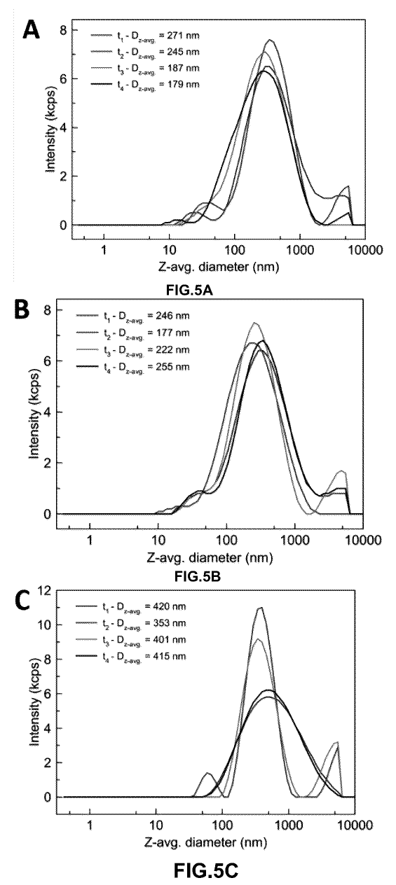

FIG. 6. Flow diagram of a microfluidic mixing chip in one embodiment thereof.

FIG. 7. Real-time dynamic light scattering of LNPs.

FIG. 8. Real-time dynamic light scattering of LNPs with increasing PEG-DMG content.

FIG. 9. Schematic overview of collection points and processing details of exemplary microfluidic chip.

FIGS. 10A-10D. (FIG. 10A, FIG. 10B, FIG. 10C, FIG. 10D) Dynamic light scattering results for DDAB/DSPC/CHOL/PEG-DMG (50/10/30/10) collected under various conditions.

FIG. 11. RiboGreen™ RNA standard curve for encapsulation studies.

FIG. 12. Dynamic light scattering of diluted LNPs after reconstitution. LNPs had a hydrodynamic diameter of 161 nm before dilution and lyophilization.

FIGS. 13A-13B. (FIG. 13A, FIG. 13B) Cryogenic electron microscopy images of LNPs with encapsulated RNA.

FIG. 14. Dynamic light scattering results for LNPs prepared at higher throughput.

FIG. 15. Modified droplet chip containing a meander mixing channel, aka convoluted mixing channel.

FIG. 16. Dynamic light scattering of LNPs prepared using droplet microchips containing straight or meander mixing channels.

FIGS. 17A-17B. Optical microscope images of microfluidic mixing of ethanolic Nile Red and aqueous methylene blue channels. (FIG. 17A) no filter (FIG. 17B) Sobel filter to enhance edges. Qₜₒₜₐₗ=299 μL/min; 75 vol. % EtOH.

FIG. 18A-18B. (FIG. 18A) Image of microchips whereby the distance between the mixing or inlet junction and meander channel is varied. (FIG. 18B) Dynamic light scattering results of particles obtained using microchips A-C.

FIGS. 19A-19B. (FIG. 19A) Illustration of droplet microchips A-B with varying meander channel length (top) and (FIG. 19B) dynamic light scattering results of particles with (green) and without (blue) PEG-DMG (bottom).

FIG. 20. Dynamic light scattering results of lipid nanoparticles prepared with varying amounts of PEG-DMG as a co-surfactant.

FIG. 21. Dynamic light scattering results of lipid nanoparticles prepared with varying amounts of PEG-DMG as a co-surfactant.

FIG. 22. Optical profilometry images showing 3D surface profile of inlet (A) and meander (B) channels.

FIG. 23. Schematic of the raster pattern (in blue) of the laser during etching of the microfluidic chips. The laser motion is aligned with the straight channels during etching.

FIG. 24. Dynamic light scattering results of lipid nanoparticles prepared as a function of volumetric throughput.

FIGS. 25A-25D. Evaluating particle size as a function microchip design: (FIG. 25A) droplet chip, (FIG. 25B) hairpin droplet chip, (FIG. 25C) meander channel droplet chip, and (FIG. 25D) dynamic light scattering results of lipid nanoparticles prepared as a chip design.

FIGS. 26A-26E. Evaluating particle size as a function microchip design: hairpin chips with varying mixing channel length and distance between hairpin turns (FIG. 26A-FIG. 26D), and (FIG. 26E) dynamic light scattering results of lipid nanoparticles prepared as a function of chip design.

FIGS. 27A-27B. (FIG. 27A) Evaluating particle size as a function microchip design: hairpin chips with varying mixing channel length and distance between hairpin turns and inlet junction (A-E), and (F) dynamic light scattering results of lipid nanoparticles prepared as a function of chip design. (FIG. 27B) Evaluating particle size as a function microchip design: hairpin chips with varying mixing channel length and distance between hairpin turns and inlet junction (A-E), and (F) dynamic light scattering results of lipid nanoparticles prepared as a function of chip design.

### DETAILED DESCRIPTION OF THE INVENTION

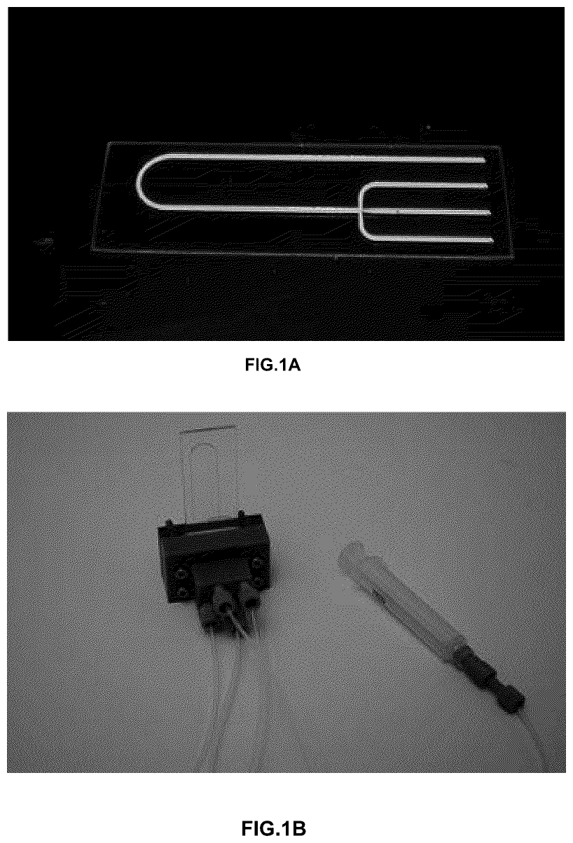

The invention includes a novel microfluidic mixing chip configured for the production of LNPs, and in particular LNPs encapsulating oligonucleotides, such as mRNA that may be used in various therapeutic applications such as vaccines and the like. The microfluidic mixing chip (1) of the invention may include a plurality of fluid channels along the surface of the chip that are configured to receive a lipid solution, optionally containing a quantity of oligonucleotides, and preferably mRNA oligonucleotides, and form LNPs that are configured to encapsulate said mRNAs. As generally shown in FIG. 1, in one preferred embodiment, a microfluidic mixing chip (1) may include a glass chip having a plurality of laser-etched fluid channels along the surface of the chip. Naturally, such example is exemplary only as other materials and methods of making microfluidic chips known in the art may be employed.

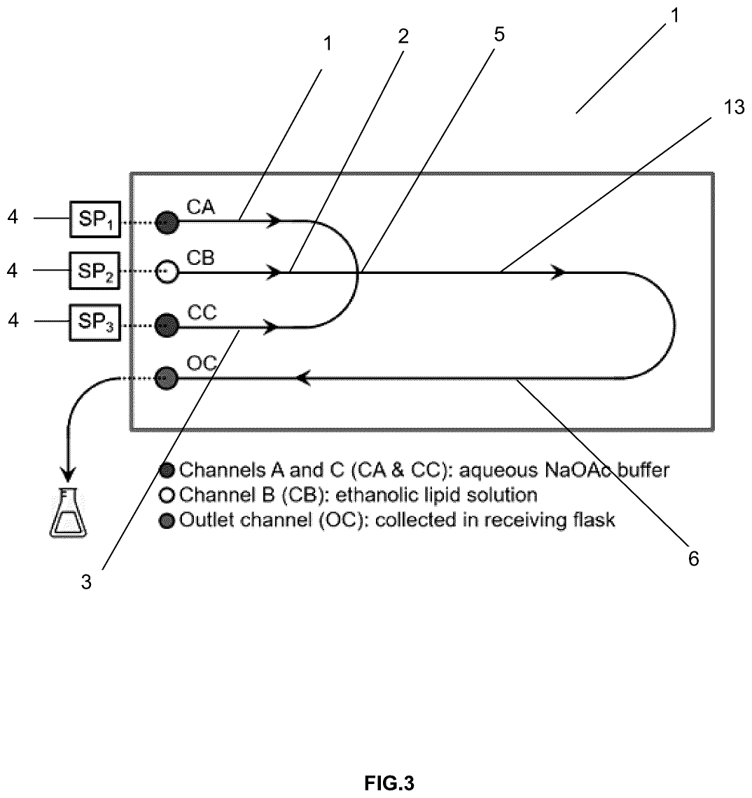

Referring now to FIG. 3, a microfluidic mixing chip (1) may include at least one lipid insertion channel (2) configured to direct a flow of a lipid solution containing a quantity of oligonucleotides. The microfluidic mixing chip (1) of the invention may further include one or more buffer channels (3) configured to direct a flow of a buffer solution, such as an aqueous NaOAc buffer solution and the like. Notably, these channels (2,3) may be in fluid communication with a reservoir of lipid solution and buffer, respectively. In this embodiment, a pump (4), which may include a mechanical or manual pump device may direct a quantity of lipid solution and buffer, respectively into their respective channels.

As detailed below, one or more pumps (4) may generate a volumetric throughput (Qₜₒₜₐₗ) through the channels of the microfluidic mixing chip (1). In one preferred embodiment, Qₜₒₜₐₗ of fluid directed through the microfluidic mixing chip (1) may be 1000 μL/min to 8000 μL/min, and preferably 2000 μL/min. Notably, the volumetric throughput (Qₜₒₜₐₗ) may affect LNP formation and size, and as such, the volumetric throughput (Qₜₒₜₐₗ) may be adjusted as desired to achieve the desired output and size of LNPs.

The microfluidic mixing chip (1) of the invention may further include one or more inlet junction(s) (5) positioned at the intersection of the lipid insertion and buffer channels (2,3), respectively. In this embodiment, lipid solution and buffer solution may enter their respective channels and be directed to the inlet junction (5) where they are mixed and directed through a single continuing mixing channel (13) facilitating the formation of LNPs. As detailed in the schematic representation of FIG. 3, the mixed mixing channel (13) may be coupled with an outlet channel (6) that may be configured to direct the now formed LNPs to a collection container or reservoir. In certain embodiments a cryoprotectant may be added to the LNP, for example trehalose or sucrose. In still further embodiment, LNPs produced by the methods and apparatus of the invention can be lyophilized and reconstituted while demonstrating enhanced retention of oligonucleotide.

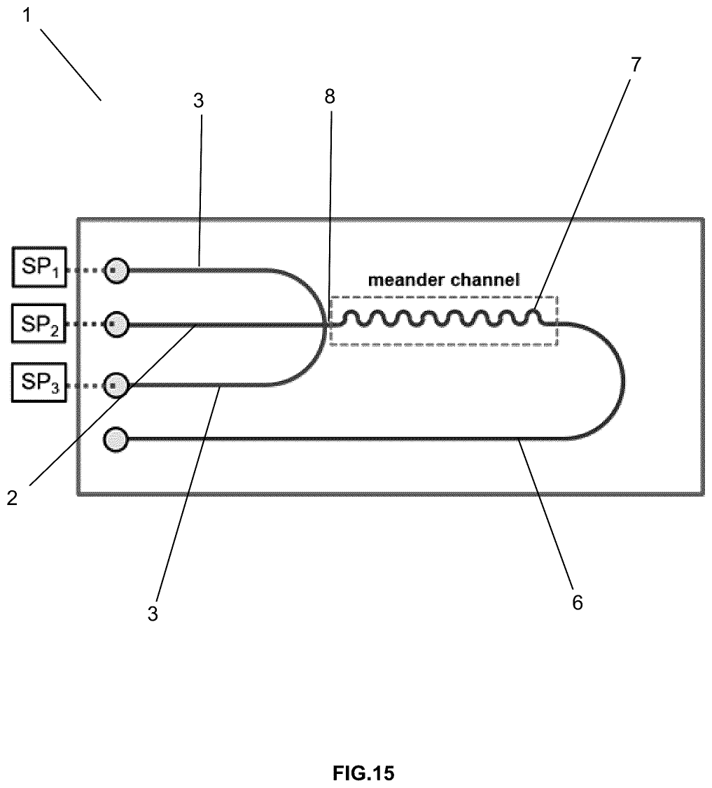

The microfluidic mixing chip (1) of the invention may further include one or more meander channel(s) (7). In the embodiment shown in FIG. 15, a meander channel (7) may include a convoluted pathway that may, in this embodiment be integrally coupled with the mixing channel (13). As detailed below, in a preferred embodiment, meander channel (7) may include a series of convoluted turns causing the mixed lipid solution and buffer to travel an extended convoluted pathway. In this embodiment, the formation of LNPs may be enhanced. For example, in one embodiment, the meander channel (7) of the invention may be configured to increase the extent of microfluidic mixing of the lipid solution and buffer during nanoparticle formation. Further, the meander channel (7) of the invention may be configured to produce LNPs having a smaller particle size and increased encapsulation of oligonucleotides.

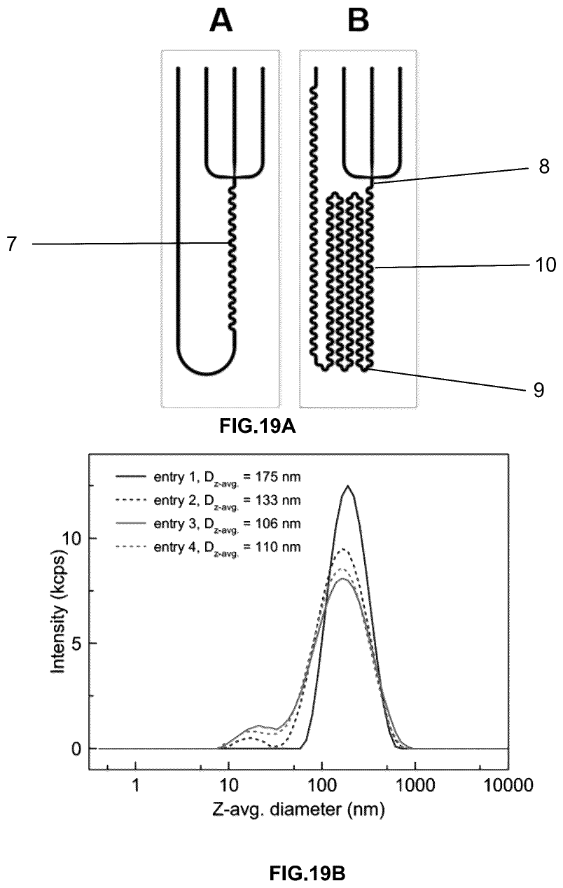

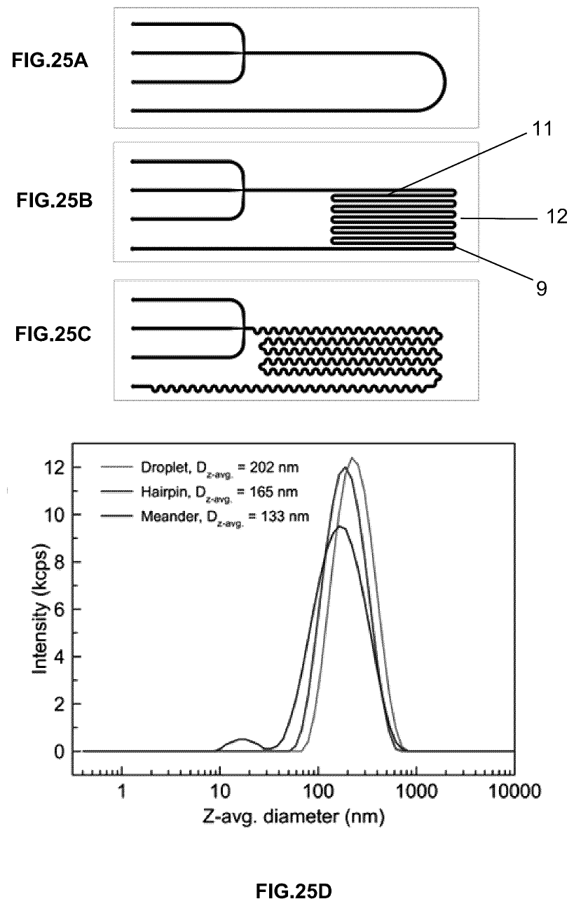

Additional embodiments include alternative configuration of the microfluidic mixing ship (1) of the invention. For example, as shown in FIGS. 15 and 25-27, the distance between the inlet junction (5) and meander channel (7), may include a variable onset length (8) which may affect LNP formation and oligonucleotide encapsulation by reducing the distance the mixed solutions travel from the inlet junction (5) prior to be directed through a first meander channel (7). As further shown in FIGS. 19 and 25, in certain preferred embodiments, the microfluidic mixing ship (1) of the invention may include a series of meander channels (7), which may be positioned in a longitudinal adjacent configuration forming a meander mixing segment (10)

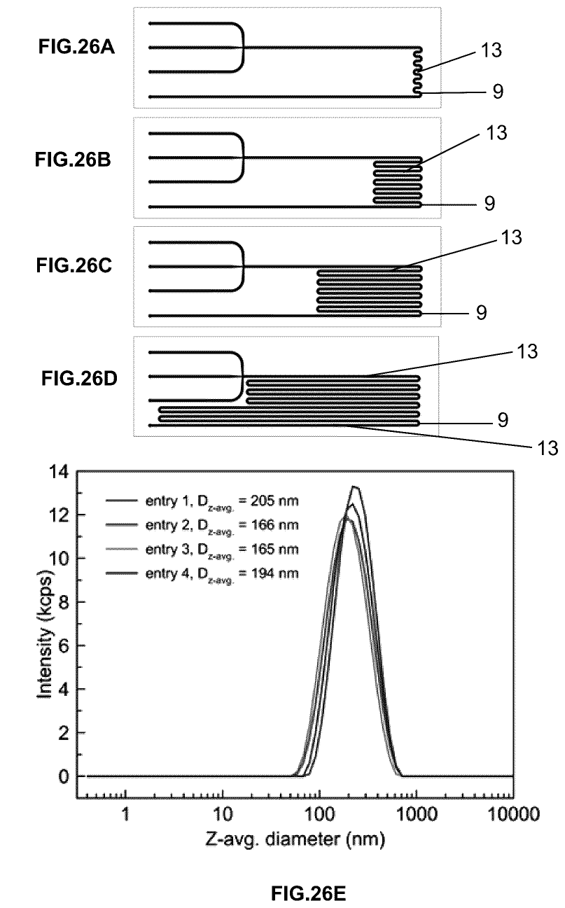

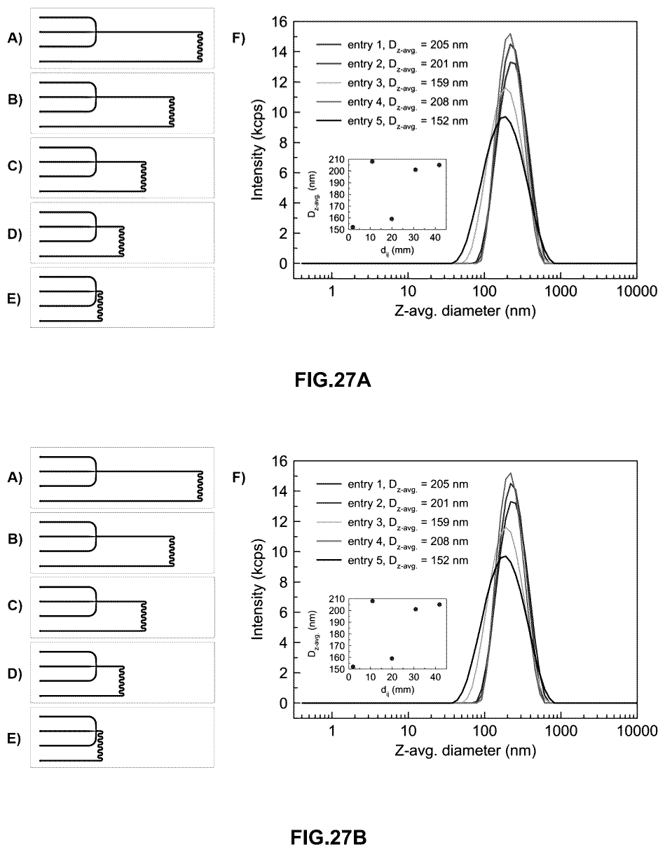

The microfluidic mixing ship (1) of the invention may further include one or a series of hairpin turns (9), which may, or may not be coupled with a meander channel (7) of the invention. As shown in FIGS. 25-27, in one embodiment, a plurality of hairpin turns (7) may be positioned adjacent one with another and may be configured to increase the extent of microfluidic mixing of the lipid solution and buffer during nanoparticle formation produced LNPs having a smaller particle size and increased encapsulation of oligonucleotides. As also shown in in FIGS. 26-27, the number and distance between hairpin turns (dm) (12) may also be variable, and include anywhere from 1 to 6 or more hairpin turns.

Notably, as shown in FIG. 26, in various embodiments the onset length (8), in this instance the distance between the inlet junction (5) and a first hairpin turn (9) may be adjusted. The plurality of hairpin turns (9) may be direct fluid through a plurality of mixing channel (13). As further shown in FIGS. 26-27, the length of the mixing channel (13), generally referred to as mixing channel length (dₘᵢₓ) (11) may be variable, such that a microfluidic mixing chip (1) having a longer mixing channel length (dₘᵢₓ) (11) may be configured to increase the extent of microfluidic mixing of the lipid solution and buffer during nanoparticle formation produce LNPs having a smaller particle size and increased encapsulation of oligonucleotides.

In one preferred embodiment, a lipid nanoparticle (LNPs) generated by the methods and apparatus of the invention comprise: (a) at least one oligonucleotide, and optionally an mRNA, optionally comprised by the (pharmaceutical) composition or vaccine as defined herein, (b) a cationic lipid, (c) an aggregation reducing agent or cosurfactant (such as polyethylene glycol (PEG) lipid or PEG-modified lipid), (d) optionally a non-cationic lipid (such as a neutral lipid), and (e) optionally, a sterol. In the context of the present invention, the term “lipid nanoparticle”, also referred to as “LNP”, is not restricted to any particular morphology, and includes any morphology generated when a cationic lipid and optionally one or more further lipids are combined, e.g., in an aqueous environment and/or in the presence of an oligonucleotide, such as an mRNA. For example, a liposome, a lipid complex, a lipoplex, an emulsion, a micelle, a lipidic nanocapsule, a nanosuspension and the like are within the scope of a lipid nanoparticle (LNP).

In one preferred embodiment, the lipid solution of the invention includes, in addition to the at least one mRNA, (i) at least one cationic lipid; (ii) a neutral lipid; (iii) a sterol/lipid; and (iv) a cosurfactant. In still further embodiments, the lipid solution of the invention includes, in addition to the at least one mRNA, (i) at least one cationic lipid; (ii) a neutral lipid; (iii) a sterol/lipid; and (iv) a cosurfactant in a molar ratio of about 50% cationic lipid: 10% neutral lipid: 39% sterol/lipid; 1% cosurfactant.

As detailed below, in still further embodiment, the lipid solution of the invention includes, in addition to at least one mRNA, a cationic lipid comprising dimethyldioctadecylammonium bromide (DDAB), a neutral lipid comprising 1,2-distearoyl-sn-glycero-3-phosphocholine (DSPC), a sterol/lipid comprising cholesterol (CHOL), and a cosurfactant comprising 1,2-dimyristoyl-rac-glycero-3-methoxypolyethylene glycol (PEG-DMG). In a preferred embodiment, the lipid solution of the invention includes, in addition to at least one mRNA, a cationic lipid comprising dimethyldioctadecylammonium bromide (DDAB), a neutral lipid comprising 1,2-distearoyl-sn-glycero-3-phosphocholine (DSPC), a sterol/lipid comprising cholesterol (CHOL), and a cosurfactant comprising 1,2-dimyristoyl-rac-glycero-3-methoxypolyethylene glycol (PEG-DMG), in an ethanol solution, in a molar ratio of about 50% DDAB: 10% DSPC: 39% CHOL; 1% PEG-DMG.

In additional embodiment, the cationic lipid may be, for example, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Ci), 1,2-Dilinoleoyi-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-DMA), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), or any combination of any of the foregoing. Other cationic lipids include, but are not limited to, N,N-distearyl-N,N-dimethylammonium bromide (DDAB), 3P—(N—(N′,N′-dimethylaminoethane)-carbamoyl)cholesterol (DC-Choi), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and LIPOFECTAMINE (comprising DOSPA and DOPE, available from GIBCO/BRL). Other suitable (cationic) lipids are disclosed in WO2009/086558, WO2009/127060, WO2010/048536, WO2010/054406, WO2010/088537, WO2010/129709, WO2011/153493, US2011/0256175, US2012/0128760, US2012/0027803, and U.S. Pat. No. 8,158,601. In that context, the disclosures of WO2009/086558, WO2009/127060, WO2010/048536, WO2010/054406, WO2010/088537, WO2010/129709, WO2011/153493, US2011/0256175, US2012/0128760, US2012/0027803, and U.S. Pat. No. 8,158,601 are incorporated herewith by reference. In some aspects the lipid may be selected from the group consisting of 98N12-5, C12-200, and ckk-E12.

The cationic lipid may also be an amino lipid. Suitable amino lipids include those having alternative fatty acid groups and other dialkylamino groups, including those in which the alkyl substituents are different (e.g., N-ethyl-N-methylamino-, and N-propyl-N-ethylamino-). In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C14 to C22 may be used. Other scaffolds can also be used to separate the amino group and the fatty acid or fatty alkyl portion of the amino lipid. Representative amino lipids include, but are not limited to, 1,2-dilinoleyoxy-3-(dimethylamino)acetoxypropane (DLin-DAC), 1,2-dilinoleyoxy-3 morpholinopropane (DLin-MA), 1,2-dilinoleoyl-3-dimethylaminopropane (DLinDAP), 1,2-dilinoleylthio-3-dimethylaminopropane (DLin-S-D A), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); C3 (US20100324120).

In some embodiments, amino or cationic lipids have at least one protonatable or deprotonatable group, such that the lipid is positively charged at a pH at or below physiological pH (e.g., pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of the lipids be present in the charged or neutral form. Lipids that have more than one protonatable or deprotonatable group, or which are zwitterionic, are not excluded from use in the invention. In some embodiments, the protonatable lipids have a pKa of the protonatable group in the range of about 4 to about 11, e.g., a pKa of about 5 to about 7. LNPs can include two or more cationic lipids. The cationic lipids can be selected to contribute different advantageous properties. For example, cationic lipids that differ in properties such as amine pKa, chemical stability, half-life in circulation, half-life in tissue, net accumulation in tissue, or toxicity can be used in the LNP. In particular, the cationic lipids can be chosen so that the properties of the mixed-LNP are more desirable than the properties of a single-LNP of individual lipids.

In certain embodiments, the LNP comprises one or more additional lipids which stabilize the formation of particles during their formation.

In some embodiments, non-cationic may be used. The non-cationic lipid can be a neutral lipid, an anionic lipid, or an amphipathic lipid. Neutral lipids, when present, can be any of a number of lipid species which exist either in an uncharged or neutral zwitterionic form at physiological pH. Such lipids include, diacylphosphatidylcholine, diacylphosphatidylethanolamine, ceramide, sphingomyelin, dihydrosphingomyelin, cephalin, and cerebrosides. The selection of neutral lipids for use in the particles described herein is generally guided by consideration of, e.g., LNP size and stability of the LNP in the bloodstream. Preferably, the neutral lipid is a lipid having two acyl groups (e.g., diacylphosphatidylcholine and diacylphosphatidylethanolamine). In some embodiments, the neutral lipids contain saturated fatty acids with carbon chain lengths in the range of C10 to C20. In other embodiments, neutral lipids with mono- or di-unsaturated fatty acids with carbon chain lengths in the range of C10 to C20 are used. Additionally, neutral lipids having mixtures of saturated and unsaturated fatty acid chains can be used. Suitable neutral lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), dimyristoyl phosphatidylcholine (DMPC), distearoyl-phosphatidyl-ethanolamine (DSPE), SM, 16-0-monomethyl PE, 16-O-dimethyl PE, 18-1-trans PE, 1-stearoyl-2-oleoyl-phosphatidyethanolamine (SOPE), cholesterol, or a mixture thereof. Anionic lipids suitable for use in LNPs include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids. In one embodiment, the neutral lipid is 1,2-distearoyl-sn-glycero-3phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1, and preferably 5:1. Amphipathic lipids refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, paimitoyloleoyl phosphatdylcholine, phosphatidylcholine, lysophosphatidylethanolamine, dipalmitoylphosphatidylcholine, dioleoylphosphatidylcholine, distearoylphosphatidylcholine, or dilinoleoylphosphatidylcholine. Other phosphorus-lacking compounds, such as sphingolipids, glycosphingolipid families, diacylglycerols, and beta-acyloxyacids, can also be used.

In some embodiments, the non-cationic lipid is present in a ratio of from about 5 mol % to about 90 mol %, about 5 mol % to about 10 mol %, about 5, 10, 15, 20, 25, 30, 35, 40, 45, 50, 55, 60, 65, 70, 75, 80, 85, or about 90 mol % of the total lipid present in the LNP. In some embodiments, LNPs comprise from about 0% to about 15 or 45% on a molar basis of neutral lipid, e.g., from about 3 to about 12% or from about 5 to about 10%. For instance, LNPs may include about 15%, about 10%, about 7.5%, or about 7.1% of neutral lipid on a molar basis (based upon 100% total moles of lipid in the LNP).

In some embodiments, a sterol/lipid may be used. The sterol is preferably cholesterol. The sterol can be present in a ratio of about 10 mol % to about 60 mol % or about 25 mol % to about 40 mol % of the LNP. In some embodiments, the sterol is present in a ratio of about 10, 15, 20, 25, 30, 35, 40, 45, 50, 55, or about 60 mol % of the total lipid present in the LNP. In other embodiments, LNPs comprise from about 5% to about 50% on a molar basis of the sterol, e.g., about 15% to about 45%, about 20% to about 40%, about 48%, about 40%, about 39% about 38.5%, about 35%, about 34.4%, about 31.5% or about 31% on a molar basis (based upon 100% total moles of lipid in the LNP).

In some embodiments, an aggregation reducing agent or cosurfactant may be employed. The aggregation reducing agent can be a lipid capable of reducing aggregation. Examples of such lipids include, but are not limited to, polyethylene glycol (PEG)-modified lipids such as PEG-DMG, monosialoganglioside Gml, and polyamide oligomers (PAO) such as those described in U.S. Pat. No. 6,320,017, which is incorporated by reference in its entirety. Other compounds with uncharged, hydrophilic, steric-barrier moieties, which prevent aggregation during formulation, like PEG, Gml or ATTA, can also be coupled to lipids. ATTA-lipids are described, e.g., in U.S. Pat. No. 6,320,017, and PEG-lipid conjugates are described, e.g., in U.S. Pat. Nos. 5,820,873, 5,534,499, 5,885,613, US20150376115A1 and WO2015/199952, each of which is incorporated by reference in its entirety.

The aggregation reducing agent or cosurfactant may be, for example, selected from a polyethyleneglycol (PEG)-lipid including, without limitation, a PEG-diacylglycerol (DAG), a PEG-dialkylglycerol, a PEG-dialkyloxypropyl (DAA), a PEG-phospholipid, a PEG-ceramide (Cer), or a mixture thereof (such as PEG-Cer14 or PEG-Cer20). The PEG-DAA conjugate may be, for example, a PEG-dilauryloxypropyl (C12), a PEG-dimyristyloxypropyl (C14), a PEG-dipalmityloxypropyl (C16), or a PEG-distearyloxypropyl (C18). Other pegylated-lipids include, but are not limited to, polyethylene glycol-didimyristoyl glycerol (C14-PEG or PEG-C14, where PEG has an average molecular weight of 2000 Da) (PEG-DMG); (R)-2,3-bis(octadecyloxy)propyl-1-(methoxy polyethylene glycol)2000)propylcarbamate) (PEG-DSG); PEG-carbamoyl-1,2-dimyristyloxypropylamine, in which PEG has an average molecular weight of 2000 Da (PEG-cDMA); N-Acetylgalactosamine-((R)-2,3-bis(octadecyloxy)propyl-1-(methoxy polyethylene glycol)2000)propylcarbamate)) (GalNAc-PEG-DSG); mPEG (mw2000)-diastearoylphosphatidyl-ethanolamine (PEG-DSPE); and polyethylene glycol-dipalmitoylglycerol (PEG-DPG). In some embodiments, the aggregation reducing agent is PEG-DMG. In other embodiments, the aggregation reducing agent is PEG-c-DMA.

In various embodiments, the molar ratio of the cationic lipid to the PEGylated lipid ranges from about 100:1 to about 25:1. In a preferred embodiment, the composition of LNPs may be influenced by, inter alia, the selection of the cationic lipid component, the degree of cationic lipid saturation, the nature of the PEGylation, the ratio of all components and biophysical parameters such as its size.

In some embodiments, LNPs may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to mRNA may range from about 5:1 to about 20:1, from about 10:1 to about 25:1, from about 15:1 to about 30:1 and/or at least 30:1 or greater than 30:1. The average molecular weight of the PEG moiety in the PEG-modified lipids can range from about 500 to about 8,000 Daltons (e.g., from about 1,000 to about 4,000 Daltons). In one preferred embodiment, the average molecular weight of the PEG moiety is about 2,000 Daltons.

The concentration of the aggregation reducing agent or cosurfactant may range from about 0.1 to about 15 mol %, per 100% total moles of lipid in the LNP. In some embodiments, LNPs include less than about 3, 2, or 1 mole percent of PEG or PEG-modified lipid, based on the total moles of lipid in the LNP. In further embodiments, LNPs comprise from about 0.1% to about 20% of the PEG-modified lipid on a molar basis, e.g., about 0.5 to about 10%, about 0.5 to about 5%, about 10%, about 5%, about 3.5%, about 3%, about 2.5%, about 2%, about 1.5%, about 1%, about 0.5%, or about 0.3% on a molar basis (based on 100% total moles of lipids in the LNP). Different LNPs having varied molar ratios of cationic lipid, non-cationic (or neutral) lipid, sterol (e.g., cholesterol), and aggregation reducing agent (such as a PEG-modified lipid) on a molar basis (based upon the total moles of lipid in the lipid nanoparticles).

The total amount of nucleic acid, particularly the one or more RNAs in the lipid nanoparticles varies and may be defined depending on the e.g., RNA to total lipid w/w ratio. In one embodiment of the invention the RNA to total lipid ratio is less than 0.06 w/w, preferably between 0.03 w/w and 0.04 w/w, or greater than 0.04 w/w/.

In some embodiments, LNPs occur as liposomes or lipoplexes as described in further detail below. In some embodiments, LNPs have a median diameter size of from about 50 nm to about 300 nm, such as from about 50 nm to about 250 nm, for example, from about 50 nm to about 200 nm, preferably 100 nm. In some embodiments, smaller LNPs may be used. Such particles may comprise a diameter from below 0.1 μm up to 100 nm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μm, less than 950 μm, less than 975 μm, In another embodiment, nucleic acids may be delivered using smaller LNPs which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nm, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm. In some embodiments, the LNP may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In other embodiments, LNPs have a single mode particle size distribution (i.e., they are not bi- or poly-modal). LNPs, as used herein may further comprise one or more lipids and/or other components in addition to those mentioned above.

As noted above, in one embodiment a LNP of the invention may encapsulate a mRNA that may indue an immune response. As such, the LNPs of the invention may include a pharmaceutical compositions as defined herein, and preferably a vaccine. A “vaccine” is typically understood to be a prophylactic or therapeutic material providing at least one epitope of an antigen, preferably an immunogen. “Providing at least on epitope” means, for example, that the vaccine comprises the epitope (or antigen comprising or providing said epitope) or that the vaccine comprises a molecule that, e.g., encodes the epitope or an antigen comprising or providing the epitope. The antigen preferably stimulates the adaptive immune system to provide an adaptive immune response. The (pharmaceutical) composition or vaccine provided herein may further comprise at least one pharmaceutically acceptable excipient, adjuvant or further component (e.g., additives, auxiliary substances, and the like). In preferred embodiments, the (pharmaceutical) composition or vaccine according to the invention comprises a plurality or more than one of the inventive mRNAs comprising a multi-valent COVID-19 mRNA vaccine as described herein. An antigen-providing mRNA in the context of the invention may typically be an mRNA, having at least one open reading frame that can be translated by a cell or an organism provided with that mRNA.

In a further embodiments, the present invention provides a composition comprising the COVID-19 mRNA vaccine of the invention, and at least one pharmaceutically acceptable carrier. In particular, the composition according to the invention comprises at least one mRNA, preferably as described herein, encoding at least one antigenic peptide or protein, and preferably a plurality of antigenic peptides or protein comprising or consisting of: 1) a spike protein (S), ii) the receptor-binding motif (RBM) of spike protein (S); and iii) an antibody (Ab) epitope identified in recovered patients proximal to the fusion peptide of S1 spike protein identified herein as FuPep, or FuPep fragment, iv) the nucleocapsid protein (NCP), as well as fragments, or variants of the same of a COVID-19 coronavirus, or from a fragment or variant of any one of these proteins, encapsulated by a LNP of the invention. The composition according to the invention is preferably provided as a pharmaceutical composition or as a vaccine.

A “therapeutically effective amount” of a compound, preferably an LNP encapsulating an oligonucleotide, and preferably an mRNA oligonucleotide of the present invention or a pharmaceutical composition thereof is an amount sufficient to provide a therapeutic benefit in the treatment of a disease or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, and/or enhances the therapeutic efficacy of another therapeutic agent. A “therapeutically effective amount” may also mean “prophylactically effective amount” of a compound of the present invention is an amount sufficient to prevent a disease or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In one embodiment, a “therapeutically effective amount” can mean an amount necessary to produce an immune response.

Pharmaceutical compositions described herein can be prepared by any method known in the art of pharmacology. In general, such preparatory methods include the steps of bringing an LNP encapsulating an oligonucleotide, and preferably an mRNA oligonucleotide into association with a carrier or excipient, and/or one or more other accessory ingredients, and then, if necessary and/or desirable, shaping, and/or packaging the product into a desired single- or multi-dose unit. Pharmaceutical or nutraceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage. A “pharmaceutical composition” may include a vaccine of the invention and an agent, e.g., a carrier, that may typically be used within a pharmaceutical composition or vaccine for facilitating administering of the components of the pharmaceutical composition or vaccine to an individual.

Each publication or patent cited herein is incorporated herein by reference in its entirety.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Initial Design and Development of Microfluidic Mixing Chip

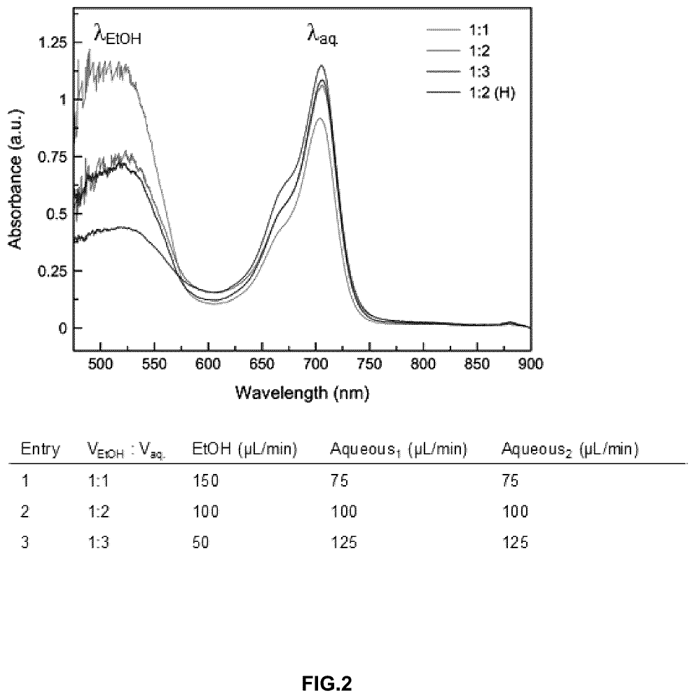

As shown in FIGS. 1-2, the present inventors fabricated a plurality of microfluidic mixing chips. The chips may be constructed of glass and may further tolerate aggressive cleaning and sterilization procedures present in GMP environments. microfluidic mixing chips underwent a series of flow tests, beginning with hand syringing fluids to ensure easy, leak-free flow. A more rigorous test included mixing dye solutions and performing real time measurements of UV visible spectroscopy after mixing. These measurements allowed the present inventors to demonstrate balanced flow between the two channels and the ability to systematically change flow rates in the individual channels. This confirms that the chips function as designed and can be used for future experiments that include reagents for making an LNP encapsulated mRNA vaccine. To perform the measurements, flow rates of ethanolic tartrazine (X_{EtOH}) and aqueous methylene blue (λ_{aq.}) were modified while keeping a constant volumetric throughput of 300 μL/min. Real-time UV-vis revealed that mixtures containing lower ethanol volume fractions resulted in a decrease in tartrazine absorbance relative to methylene blue. Furthermore, a simple hysteresis experiment showed how previously programmed flow rates (entry 2 in table) can be recovered (see 1:2 H).

### Example 2: Continuing Design and Validation of Microfluidic Mixing Chip

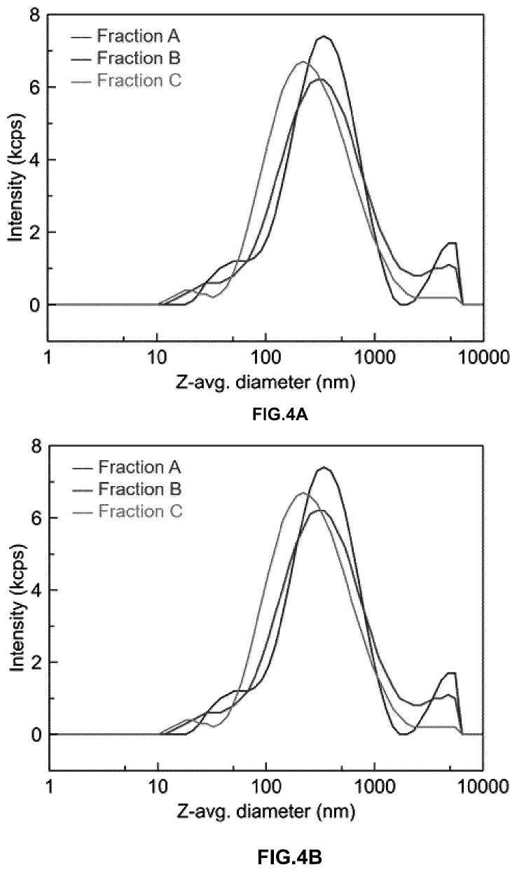

As shown in FIGS. 3-4, the present inventors fabricated additional glass microfluidic mixing chips and performed initial experiments on the formation of lipid nanoparticles using the microfluidic mixing chips. For these initial experiments, the present inventors did not use RNA but tested the nanoparticle formation only with a mixture of lipids. The lipids were dissolved in ethanol and co-injected with an aqueous buffer solution (see FIG. 3). We captured three separate aliquots of the nanoparticles during the course of a single experiment and measured their sized by dynamic light scattering (DLS). The sizes systematically trended towards smaller particles with narrower size distributions, going from 239 to 178 nm (see FIG. 4A). Given the timing of the aliquots, this indicates about a 20 minute equilibration time before the particle size stabilizes near the target size and with low size dispersity. A period of variation at the beginning of a microfluidic process is not unheard of and can be mitigated by a number of means, including the use of a back pressure regulator.

In this experiment, the lipid formulation comprised Dimethyldioctadecylammonium bromide (DDAB) cationic lipid/surfactant, 1,2-distearoyl-sn-glycero-3-phosphocholine (DSPC) a neutral/phopholipid, and cholesterol (CHOL) a sterol/lipid were combined with ethanol to prepare a 50 mM solution at a molar ratio of 50/10/40 (DDAB/DSPC/CHOL) which was then connected to a syringe pump (FIG. 3, SP₂). Lipid nanoparticles were formed upon microfluidic mixing with aqueous sodium acetate buffer (FIG. 3, CA/CC) at a 3:1 water/ethanol volumetric ratio and collected as 2-mL fractions to evaluate nanoparticle size as a function of time (FIG. 4A). A significant decrease in the Z-avg. particle diameter and polydispersity was observed between initial and final fractions (239 v. 178 nm, respectively), indicating a 20-minute equilibration period may be needed to obtain monodisperse LNPs.

### Example 3: Calibration of Upper-Limits of Volumetric Outputs

The present inventors investigated the effect of volumetric throughput on particle size in order to find upper limit conditions of potential scale-up. Lipid nanoparticles (LNPs) were formed upon microfluidic mixing with aqueous sodium acetate buffer (FIG. 3, CA/CC) at a 3:1 water/ethanol volumetric ratio and collected as 2-mL fractions to evaluate nanoparticle size as a function of time (FIG. 4B). Volumetric throughput was increased from 300 μL/min to 400 μL/min, and the obtained LNPs displayed a slight increase in size as compared to previous attempts (200 nm v. 180 nm, respectively). Furthermore, a uniform particle size was obtained relatively quickly as compared to previous attempts at lower throughputs. In order to better validate throughput-dependent trends in LNP size, a new glass microchip was manufactured and designed to have an increased volumetric capacity (i.e., twice the channel depth as compared to previously used microchips) through a modified laser engraving step and will be used in future studies targeting higher throughputs.

### Example 4: Determination of Effect of Volumetric Throughput on LNP Size

The present inventors further investigated the effect of volumetric throughput on particle size. Lipid nanoparticles (LNPs) were formed upon microfluidic mixing with aqueous sodium acetate buffer (FIG. 3, CA/CC) at a 25 vol. % ethanolic volume fraction and collected as 2-mL aliquots to evaluate nanoparticle size as a function of time. Volumetric throughput was increased from 298 μL/min (Table 1, entry 1) to 600-2000 μL/min (Table 1, entries 2-4), and the obtained LNPs displayed a modest increase in size until reaching significantly higher throughput (Table 1, entry 4). Importantly, it was shown that high throughputs can be achieved when preparing LNPs and can potentially be used to control particle size. Future studies focusing on decreasing particle size will employ a poly(ethylene glycol) surfactant at a variety of concentrations, which has been shown previously to allow control over LNP size.

### Example 5: Real-Time Determination of LNP Size

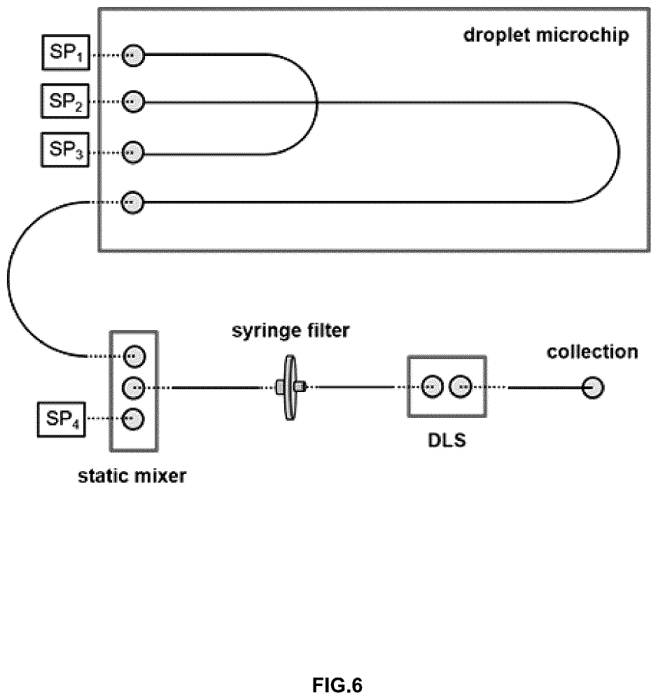

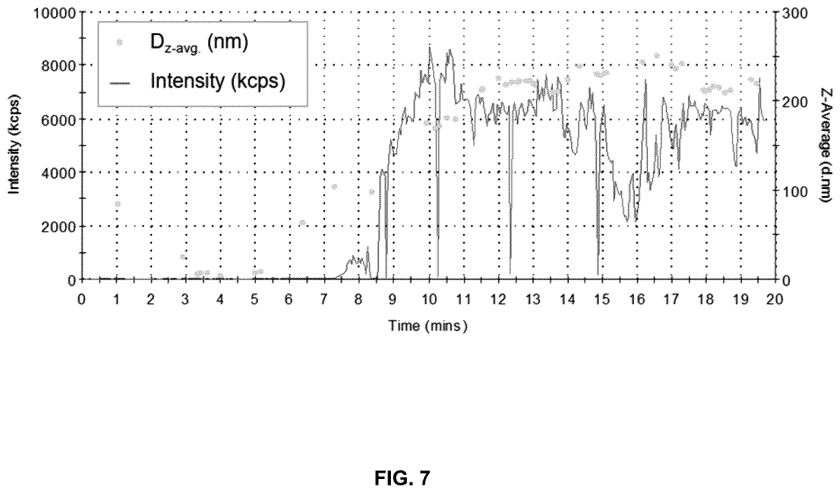

The present inventors next investigated whether nanoparticle sizes could be analyzed in real-time using a quartz flow cell connected to a dynamic light scattering detector. Lipid nanoparticles (LNPs) were formed upon microfluidic mixing of an ethanolic lipid solution with nuclease-free sodium acetate buffer (25 vol. % ethanolic volume fraction, 299 μL/min) and were subsequently diluted in flow with nuclease-free PBS (900 μL/min, SP₄), in turn producing an overall throughput of 1.2 mL/min (see FIG. 6 for schematic overview). Real-time analysis revealed equilibrium particle diameters ranging from 200-250 nm (FIG. 7). Ongoing work is focused on exploring new filtration strategies in efforts to have multi-hour size monitoring.

### Example 6: Directed Tuning of LNP Size Using Varying Concentrations of Surfactant

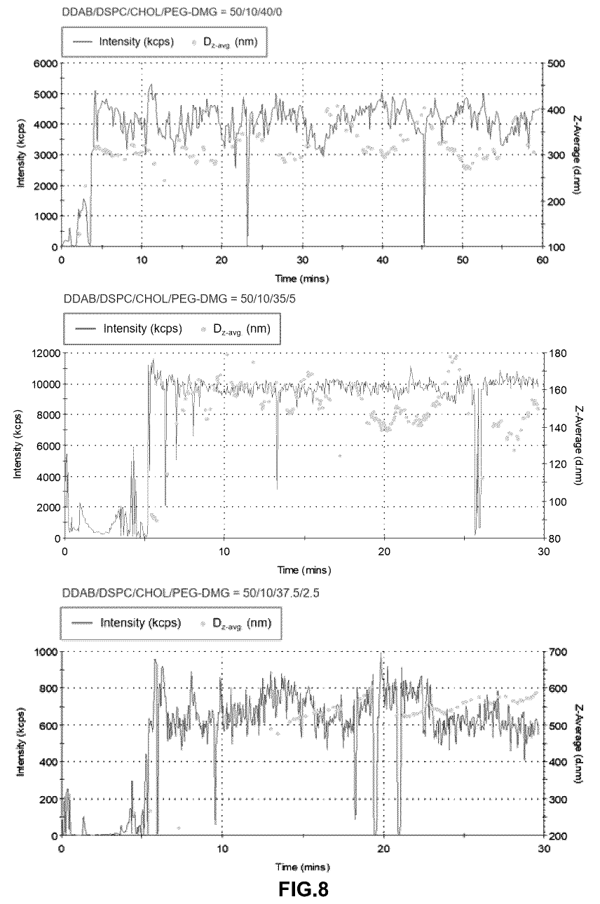

As shown in FIG. 8, the present inventors next investigated whether nanoparticle sizes could be tuned by the addition of PEG surfactant (PEG-DMG). Lipid nanoparticles (LNPs) were formed upon microfluidic mixing of an ethanolic lipid solution with nuclease-free sodium acetate buffer (25 vol. % ethanolic volume fraction, 299 μL/min) and were subsequently diluted in flow with nuclease-free PBS (900 μL/min), in turn producing an overall throughput of 1.2 mL/min. When PEG-DMG was added at 2.5 mole percent a substantial increase in size occurred. However, when higher PEG-DMG amounts were targeted (5 mole percent) a large reduction in particle size was ultimately obtained binging the average size down to about 150 nm. Future studies will explore additional PEG-DMG content to further reduce particle size to get to the desired size of 100 nm.

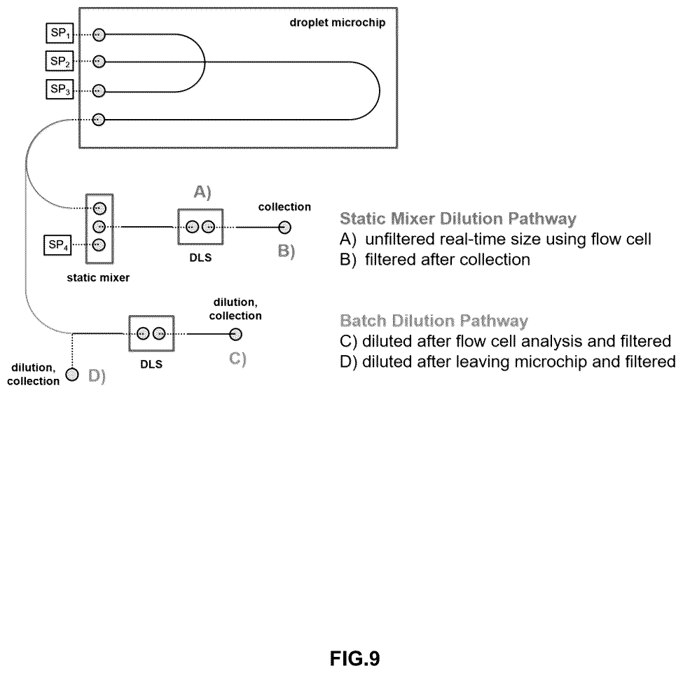

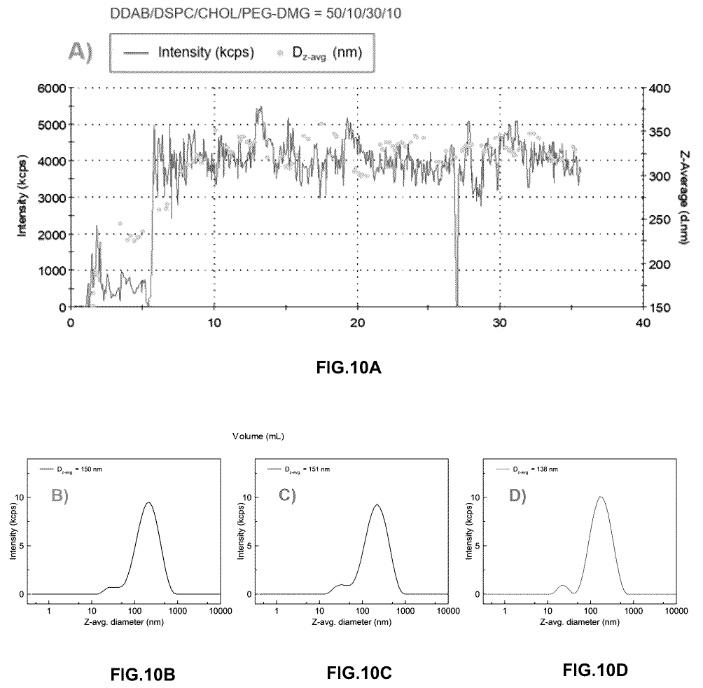

Additionally, processing conditions were modified in order to study the impact of in-flow dilution and connections to the flow cell. Lipid nanoparticles (LNPs) were formed upon microfluidic mixing of an ethanolic lipid solution with nuclease-free sodium acetate buffer (25 vol. % ethanolic volume fraction, 299 μL/min) and were subsequently diluted either after collection (3× total volume) or in flow (900 μL/min) with nuclease-free PBS. When comparing sizes between real-time analysis and after collection/filtration, a substantial difference was observed (FIG. 9, Paths AB; FIG. 10A-B). When comparing the impact of in-flow dilution using a static mixing tee to bath dilution, no difference in size was observed (FIG. 9, Paths B/C; FIG. 10B-C). This suggests this method of dilution could be used for future applications without perturbing particle size. Ultimately, when samples where diluted shortly after microfluidic mixing and avoided further travel through the DLS flow cell, small sizes (D_{z-avg.}=138 nm) were achieved (FIG. 9, Path D; FIG. 10D), making this the smallest particle size achieved to date.

### Example 7: Solid State LNP Production

The present inventors next investigated whether previously prepared lipid nanoparticles could be lyophilized and stored in the solid state in efforts to extend the lifetime of the particles as well as limit the cost of transportation. DDAB/DSPC/CHOL/PEG-DMG (50/10/30/10) particles having a size of 138 nm were lyophilized (freeze dried) and the resultant powder subsequently resuspended in water. Once the particles were resuspended, a significant increase in particle size was observed (D_{z-avg.}=251 nm). The addition of ethanol to the solution led to even larger particles (D_{z-avg.}=375 nm) which several days later had continued to increase in size (D_{z-avg.}=450 nm). The increase in size is likely caused by agglomeration which might be prevented by the addition of a drying aid to stabilize the lipid nanoparticles and separate them during the dehydration/rehydration process.

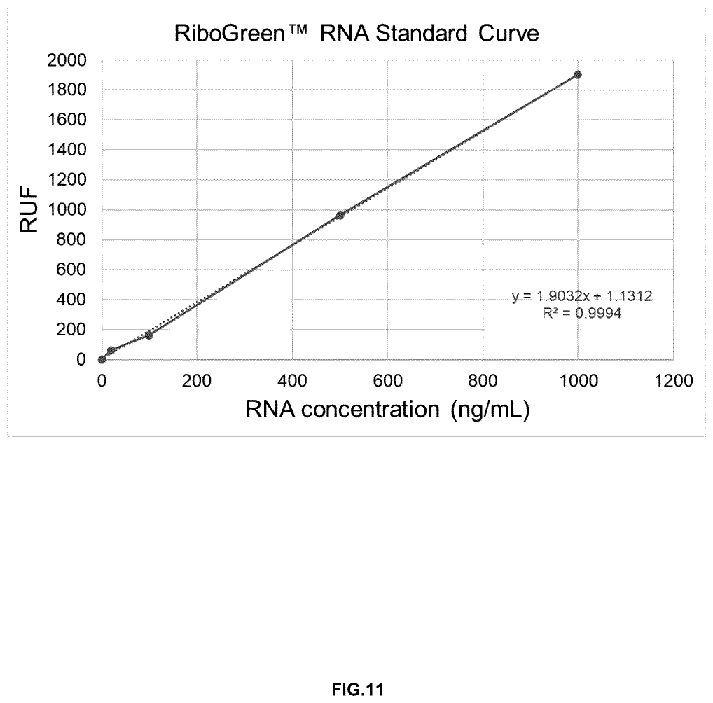

In another experiment, DDAB/DSPC/CHOL/PEG-DMG (50/10/30/10) particles (D_{z-avg.}=161 nm) were lyophilized in the presence of trehalose (20% w/v) serving as a lyoprotectant. The solution also contained phosphate buffer salts at the standard concentration. Once the particles were reconstituted with deionized water, an increase in particle size was observed (D_{z-avg.}=211 nm). This approximately 30% increase in hydrodynamic diameter is considerably lower than the 80% increase in size previously observed without trehalose addition, demonstrating a significant protective influence of the trehalose. Additionally, a standard curve was generated for RNA assays using RiboGreen™ as a fluorescent indicator (FIG. 11). This may be used to quantify encapsulation efficiency of LNPs in the presence of RNA.

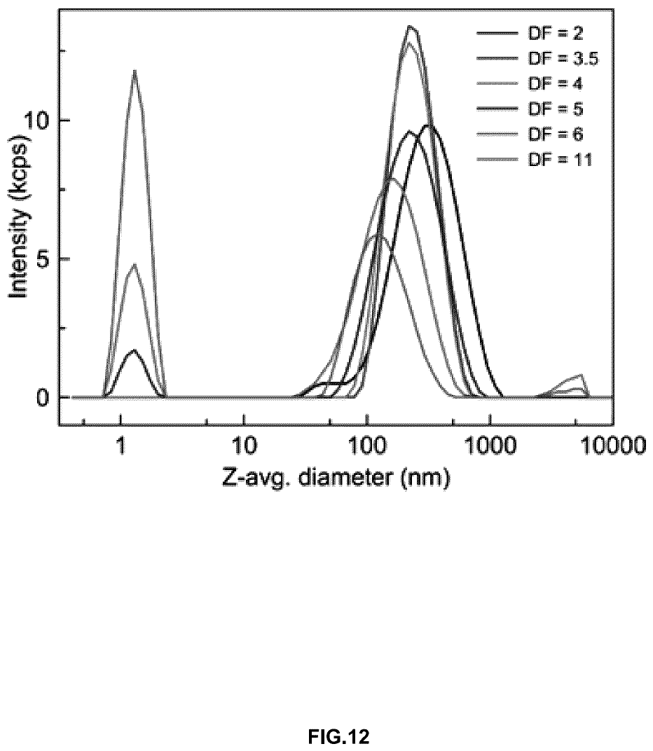

Previously the present inventors showed that the presence of trehalose during freezing, drying, and reconstituting partially mitigated the increase in size. Here we maintained the trehalose concentration at 20% w/v while diluting the sample with water. It was expected that increased water and trehalose would lessen any interactions between lipid nanoparticles during freezing, drying, and reconstitution minimizing the opportunity for size increases. DDAB/DSPC/CHOL/PEG-DMG (50/10/30/10) particles (D_{z-avg.}=161 nm) were diluted with deionized (DI) water and subsequently lyophilized in the presence of trehalose (20% w/v) serving as a cryo-(lyo)-protectant. Particles were reconstituted with DI water to the post dilution volume and characterized by dynamic light scattering to evaluate particle size (Table 2; FIG. 12).

Importantly, an inverse relationship between particle size and dilution factor was observed, and particle sizes similar to initial values could be obtained with appropriate dilution. When particles were diluted greater than a factor of four (Table 2, entries 5-7) multimodal size distributions were observed (FIG. 12), and smaller sized aggregates (D_{z-avg.}=1.3 nm) grew in relative intensity upon increased dilution.

### Example 8: Characterization of LNP Through Cryogenic Electron Microscopy

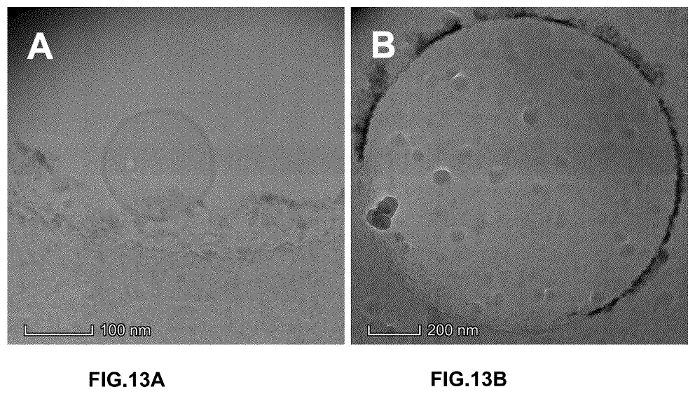

The present inventors next characterized the lipid nanoparticles (LNPs) through cryogenic electron microscopy (Cryo-EM). DDAB/DSPC/CHOL/PEG-DMG (50/10/30/10) particles (D_{z-avg.}=138 nm) were formed via microfluidic mixing with aqueous RNA from baker's yeast. Fluorescence spectroscopy revealed an encapsulation efficiency of 20% as determined by a Ribogreen™ assay before and after nanoparticle digestion using Triton X-100. Cryo-EM images (FIG. 13) show evidence of liposomal morphology, and particle sizes are consistent with values obtained by dynamic light scattering.

### Example 9: Directed Reduction of LNP Size Utilizing Microfluidic Parameter

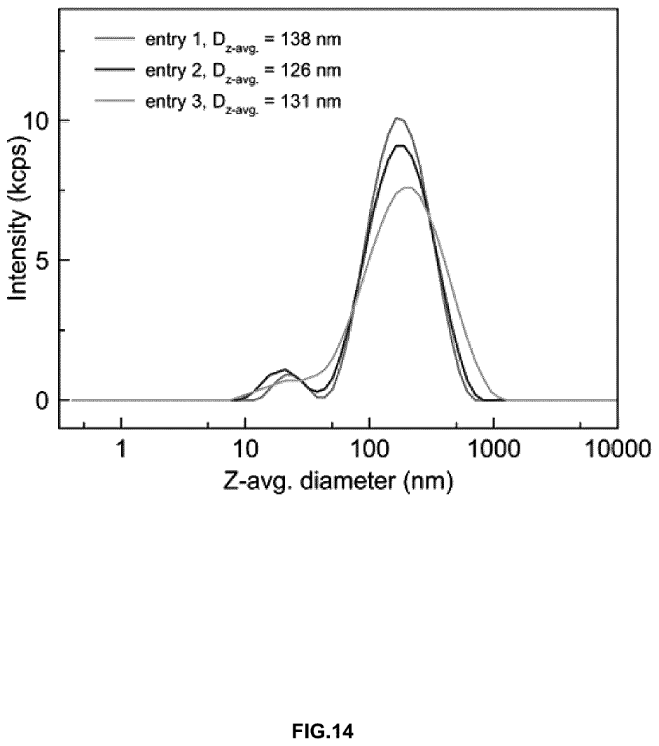

As shown in FIG. 14, the present inventors investigates parameters which may lead to a reduction in particle size of lipid nanoparticles (LNPs). DDAB/DSPC/CHOL/PEG-DMG (50/10/30/10) particles were formed via microfluidic mixing with aqueous RNA from baker's yeast. Previous work showed flow rates can alter LNP size so the present inventors increased volumetric throughput (Qₜₒₜₐₗ) to decrease particle size (Table 3). Importantly, a notable reduction in particle size (Table 3, entry 2) was observed when operating at high flow conditions (2000 μL/min). Fluorescence spectroscopy of particles prepared under these conditions revealed an encapsulation efficiency of 22% as determined by a Ribogreen™ assay before and after nanoparticle digestion using Triton X-100, indicating the higher flow rates do not affect encapsulation efficiency. It was also hypothesized that particle size could be further influenced by varying the volumetric flow rate ratio of aqueous and ethanol solutions. However, this only showed a slight reduction in particle size when compared to standard conditions (Table 3, entry 3).

### Example 10: Modification in Microfluidic Mixing Chip to Reduce LNP Size

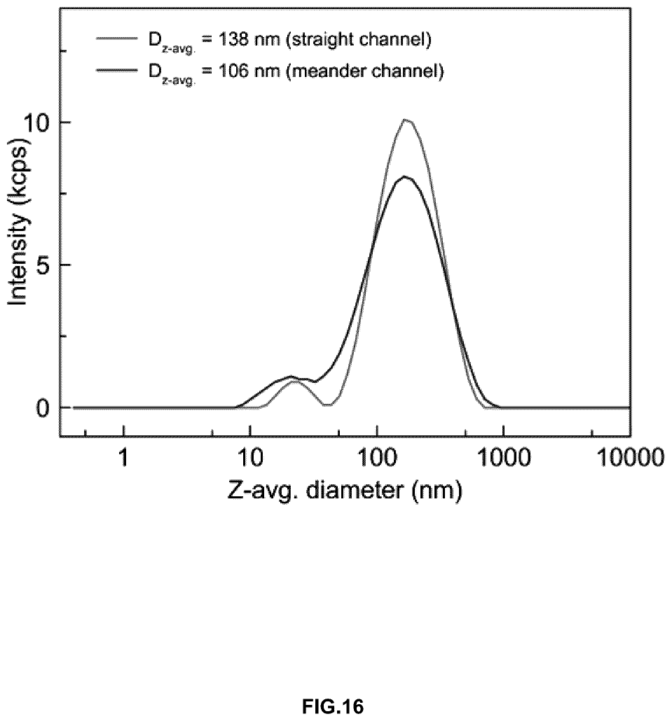

The present inventors next investigated whether modifications in microchip design would lead to a reduction in particle size of lipid nanoparticles (LNPs). Droplet microchips were modified by installing a meander channel (FIG. 15) to increase the extent of microfluidic mixing during nanoparticle formation. DDAB/DSPC/CHOL/PEG-DMG (50/10/30/10) particles were formed via microfluidic mixing with aqueous sodium acetate buffer, and volumetric throughput (Qₜₒₜₐₗ) was held at high flow conditions (2000 μL/min). A notable reduction in particle size was observed (D_{z-avg.}=106 nm, FIG. 16), leading to formation of LNPs approaching a diameter of 100 nm.

### Example 11: Characterize the RNA Encapsulation Efficiency of Lipid Nanoparticles (LNPs)

The present inventors next characterized the RNA encapsulation efficiency of lipid nanoparticles (LNPs) made using our microfluidic approach. DDAB/DSPC/CHOL/PEG-DMG (50/10/30/10) particles were formed via microfluidic mixing with aqueous RNA from baker's yeast. Fluorescence spectroscopy was used to determine encapsulation efficiency by a Ribogreen™ assay before and after nanoparticle digestion using Triton X-100. After optimization of the nanoparticle digestion conditions, a high encapsulation efficiency (71%) was observed for particles prepared under high throughput conditions (2000 μL/min). The optimization of the nanoparticle digestion indicates that previous measurements of encapsulation efficiency may have underreported the encapsulation efficiency due to incomplete digestion. Using this newly optimized approach the present inventors found that particles prepared under lower flow rates (299 μL/min) exhibited lower encapsulation efficiency (40%), which can likely be attributed to less efficient mixing during encapsulation. The present inventors next studied whether encapsulated RNA would survive lyophilization and subsequent reconstitution in the presence of trehalose. Importantly, Ribogreen™ assay of reconstituted LNPs (dilution factor=5) containing RNA showed 90% retention of encapsulated RNA.

### Example 12: Characterization of Microfluidic Mixing Chip Configuration

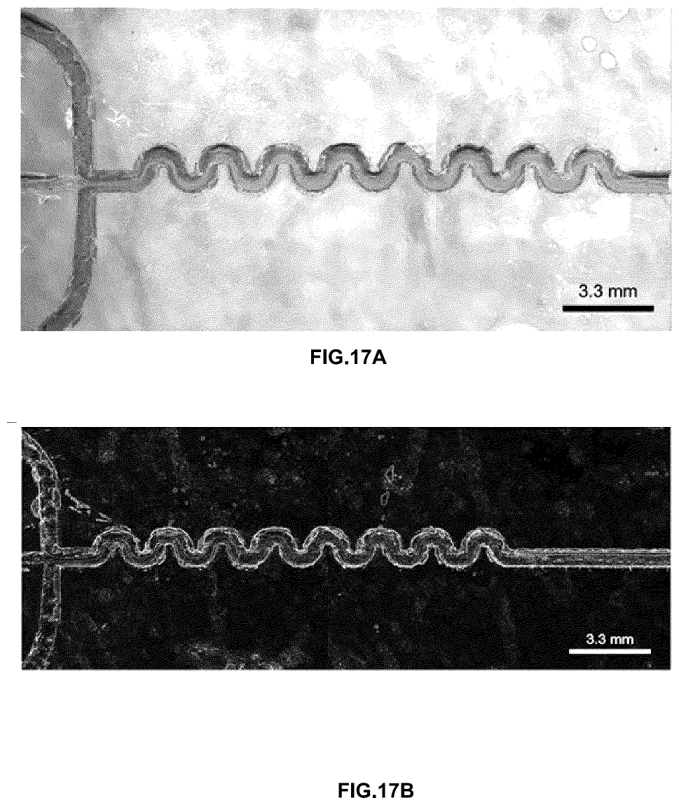

The present inventors further characterized droplet microchips by studying the mixing profile of dyed solutions in flow. An ethanolic solution containing Nile Red was mixed with aqueous methylene blue and imaged under a dissection microscope (FIG. 17). Mixing features and solvent interfaces can be observed (FIG. 17A) and further analyzed through the application of a Sobel filter (FIG. 17B) that enhances the edges between the fluid flows. Gradual widening of the ethanol channel (shown in red) is observed, indicating the formation of a more homogeneous solution.

### Example 13: Modifications in Microfluidic Mixing Chip Resulting in Reduction in LNP Size

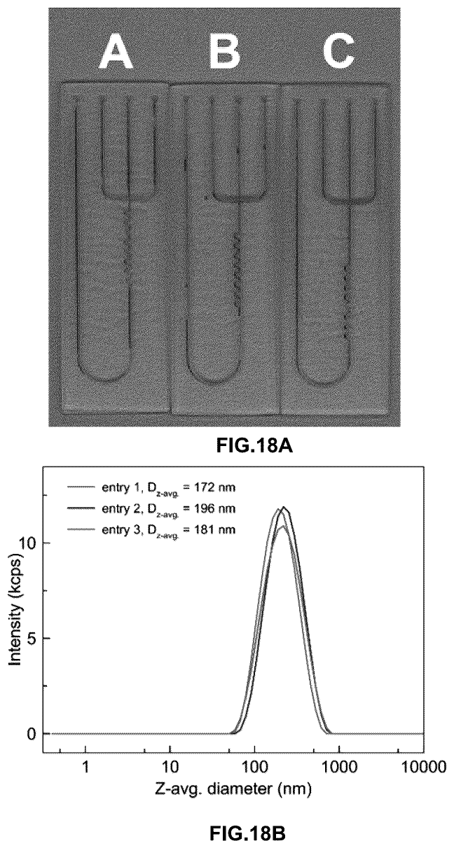

The present inventors investigated whether modifications in microchip design would lead to a reduction in particle size of lipid nanoparticles (LNPs). Droplet microchips were modified by tuning the distance between the mixing or inlet junction and meander channel, defined here as the onset length (FIG. 18A, Table 4). DDAB/DSPC/CHOL (50/10/40) particles were formed via microfluidic mixing with aqueous sodium acetate buffer, and volumetric throughput (Qₜₒₜₐₗ) was held at high flow conditions (2000 μL/min). Particle sizes varied from 172-196 nm and did not seem to correlate to differences in onset length, though the particles with the smallest sizes were achieved through the immediate entry into the meander channel. These sizes are considerably larger than sizes that we have recently reported. Previous examples have shown that the additional use of PEGylated lipid can reduce LNP sizes.

### Example 14: Modifications in Microfluidic Mixing Chip and Lipid Formulation Resulting in Reduction in LNP Size

The present inventors investigated whether modifications in microchip design and lipid composition would lead to a reduction in particle size of lipid nanoparticles (LNPs). DDAB/DSPC/CHOL/PEG-DMG (50/10/40/0 and 50/10/30/10) particles were formed via microfluidic mixing with aqueous sodium acetate buffer using a droplet microchip with varying meander channel length (FIG. 19), and volumetric throughput (Qₜₒₜₐₗ) was held at high flow conditions (2000 μL/min). As noted above, particles composed of DDAD/DSPC/CHOL exhibited a marked decrease in size (Table 5, entries 1-2) when the meander channel length was extended, presumably due to increased mixing. Although the introduction of PEG-DMG did lead to a decrease in particle size (Table 5, entries 3-4), the extension of meander channel length surprisingly did not lead to a further reduction in particle size. It appears that the PEG-DMG lipid significantly alters the mixing dynamics in a way that allows more thorough mixing with a shorter meander channel.

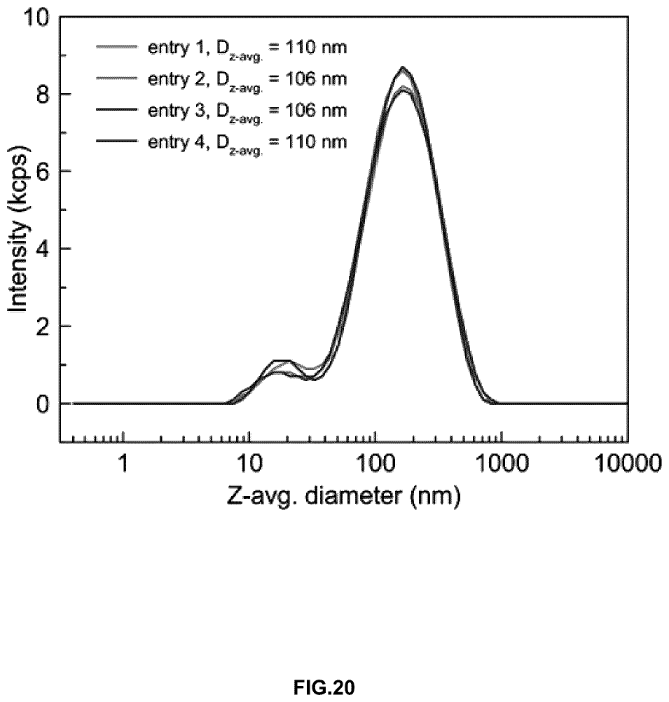

The present inventors next sought to investigate whether the amount of PEG-DMG co-surfactant could be reduced while maintaining small particle size (D_{z-avg.}=100-110 nm). DDAB/DSPC/CHOL/PEG-DMG (1-10 mol % PEG-DMG) lipid nanoparticles (LNPs) were formed via microfluidic mixing with aqueous sodium acetate buffer using a droplet microchip with an extended meander channel, and volumetric throughput (Qₜₒₜₐₗ) was held at high flow conditions (2000 μL/min). The amount of PEG-DMG was successfully reduced from 10 mol % to as little as 1 mol % without any detriment to particle size (FIG. 20, Table 6). Importantly, this leads to a significant reduction in cost upon commercialization and will allow for more experiments to be performed with our current supply of PEG-DMG.

### Example 15: Production of Sub-100 nm LNPs

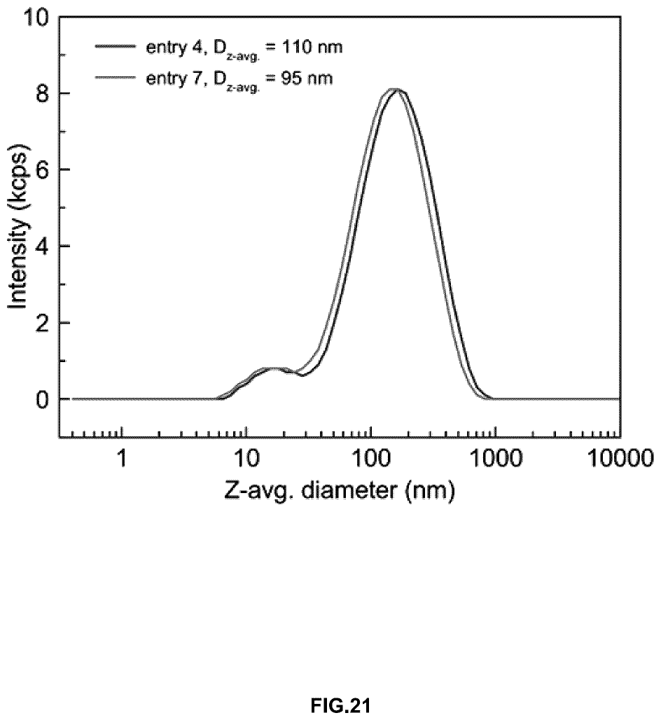

The present inventors next investigated the production of sub-100 nm lipid nanoparticles by targeting higher volumetric throughput (Qₜₒₜₐₗ) while maintaining an optimized PEG-DMG cosurfactant content (1 mol %). DDAB/DSPC/CHOL/PEG-DMG (1-10 mol % PEG-DMG) lipid nanoparticles (LNPs) were formed via microfluidic mixing with aqueous sodium acetate buffer using a droplet microchip with an extended meander channel, and volumetric throughput was held at high flow conditions (2000-4000 μL/min). When volumetric throughput was increased from 2000 μl/min to 4000 μl/min, a 15 nm reduction in particle size was observed, resulting in 95 nm LNPs (FIG. 21, Table 7, entry 7). This is beneficial in two ways. First, this is the first demonstration of the targeted size regime of 90-100 nm. Second, this doubles the throughput of the chip, approaching commercial scale production of lipid nanoparticles.

### Example 16: Characterized of Channel Dimensions of the Microfluidic Chip

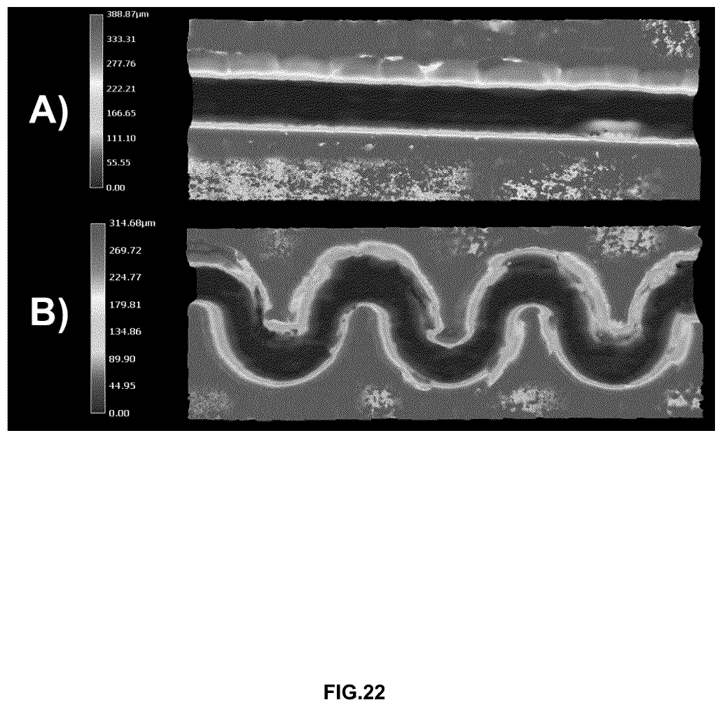

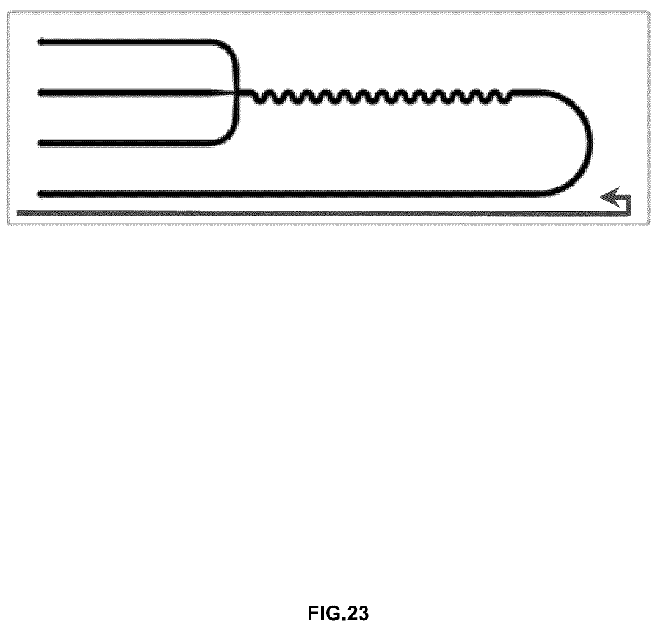

The present inventors next investigated the channel dimensions of the droplet microchips containing a meander mixing segment and analyzed microchannel topographical features using optical profilometry (FIG. 22-23). The results are summarized in Table 8. While all channels are rendered at the same width, the result of the laser etching varies between the straight and curved sections, leading to a smaller cross-sectional area in the curved sections. This is likely due to the method of laser etching, where the raster pattern leads to the pulsed laser moving in the direction of the straight channels during cutting (See FIG. 23). This constant pulsing likely leads to local heating that enhances the laser's material removal. On the other hand, when creating the curved channels, the laser is alternately pulsing and resting during the cutting which would lead to less local heating of the glass which may explain the smaller cross-sectional area.

### Example 17: Characterization of Maximum Throughput (Qₜₒₜₐₗ) Microfluidic Chips Containing an Extended Meander Channel

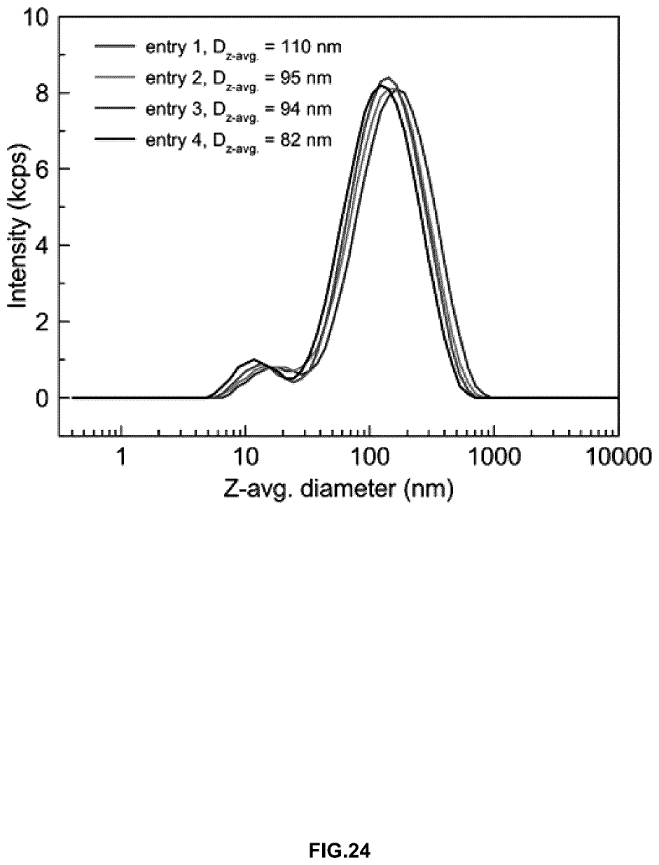

The present inventors next investigated the maximum allowable working throughput (Qₜₒₜₐₗ) of droplet microchips containing an extended meander mixing channel. DDAB/DSPC/CHOL/PEG-DMG (50/10/39/1) lipid nanoparticles (LNPs) were formed via microfluidic mixing with aqueous sodium acetate buffer, and volumetric throughput was increased until the point of syringe pump failure due to substantial backpressure. In agreement with previous results, a decrease in particle size was observed with increasing throughput (FIG. 24, Table 9), and the maximum allowable working throughput was determined to be 8000 μL/min. Additionally, the Reynolds Number (Re=196) and backpressure (3.36 psi) under these conditions (Qₜₒₜₐₗ=8000 μL/min) were able to be calculated using Poiseuille's formula for frictional pressure drop in a channel under laminar flow (Re<2000).

### Example 18: Characterization of Microfluidic Chip Design and Mixing Channel Length on of LNP Size

The present inventors next investigated how chip design and mixing channel length and shape affect the size of lipid nanoparticles (LNPs) obtained during microfluidic mixing. The mixing channel length of the original droplet chip design (FIG. 25A) was extended through the introduction of either hairpin turns (FIG. 25B) or meander mixing segments (FIG. 25C). Importantly, the hairpin-containing droplet chip was designed to have the same overall mixing channel length as the meander channel droplet chip. DAB/DSPC/CHOL (50/10/40) LNPs were formed via microfluidic mixing with aqueous sodium acetate buffer, and volumetric throughput was held at 2000 μL/min. It was found that the introduction of hairpin turns, as well as extending the mixing channel, resulted in a decrease in particle size when compared to the unmodified droplet chip (Table 10, entries 1-2, FIG. 25D). However, the introduction of meander mixing channels led to a further reduction in particle size (Table 10, entry 3).

The mixing channel length (dₘᵢₓ) and distance between hairpin turns (dim) was varied while keeping a constant number of hairpin turns (FIG. 26, A-D). DDAB/DSPC/CHOL (50/10/40) LNPs were formed via microfluidic mixing with aqueous sodium acetate buffer, and volumetric throughput was held at 2000 μL/min. We hypothesized microchip A would produce the smallest LNPs due to the low value of dim. However, it was found that the microchips B and C, which contained intermediate mixing channel lengths and distance between hairpin turns, produced smaller sized particles (Table 11, entries 2-3) when compared to microchips A and D.

The mixing channel length (dₘᵢₓ) and distance between hairpin turns and the inlet junction (dᵢⱼ) were varied while keeping a constant number of hairpin turns (FIG. 27, A-E). DDAB/DSPC/CHOL (50/10/40) LNPs were formed via microfluidic mixing with aqueous sodium acetate buffer, and volumetric throughput was held at 2000 μL/min. Microchips B and D were manufactured in effort to further correlate D_{z-avg.} and dᵢⱼ. Generally, it was observed that a reduction in di, led to an exponential decrease in the size of LNPs (FIG. 27F, inlet graph). However, particles obtained using microchip D (Table 12, entry 4) fell well outside the expected trend and warrants reevaluation.

### Example 19: Impact of Potential Cold Chain on LNP Size

The present inventors next investigated how a potential cold chain would impact the size of lipid nanoparticles (LNPs). Trehalose was chosen as a model cryoprotectant, and LNP solutions were evaluated before and after storage in either a −20 or −80° C. freezer. DDAB/DSPC/CHOL (50/10/40) LNPs were formed via microfluidic mixing with aqueous sodium acetate buffer, and volumetric throughput was held at 2000 μL/min. Particle size was first evaluated in the absence of trehalose (Table 13, entries 1-3). An increase in particle size was observed when particle solutions were frozen at −20 and −80° C., likely due to aggregation during freezing. Trehalose was then added in effort to prevent particle aggregation during cold storage. Interestingly, dynamic light scattering results showed an increase in particle size when trehalose was added either after particle collection (Table 13, entry 4) or during particle formation (Table 13, entry 5). Although particle size appeared to increase from the addition of trehalose, good retention in particle size was observed after thawing frozen trehalose-containing LNP solutions (Table 13, entries 6-7). Lastly, trehalose content was reduced from 20 to 5 wt. % in effort to minimize its effect on particle size (Table 13, entry 8). These results are significantly different from our previous experiments with using trehalose during freeze drying where we noted little size change. It is worth noting the differences between these experiments. The previous experiments were significantly diluted with water (between 2- and 11-fold dilutions) and were performed using 10% PEG-DMG in the LNPs.

The present inventors further studied how a potential cold chain would impact the size of lipid nanoparticles (LNPs). Sucrose was studied as an alternative to trehalose as a cryoprotectant, and LNP solutions were evaluated before and after flash freezing. DDAB/DSPC/CHOL/PEG-DMG (50/10/39/1) LNPs were formed via microfluidic mixing with aqueous sodium acetate buffer, and volumetric throughput was held at 2000 μL/min. Particle size was first evaluated in the absence of sucrose (Table 14, entries 1-3). An increase in particle size was observed when particle solutions were frozen in liquid nitrogen (Table 14, entry 2) as well as liquid ethane (Table 14, entry 3). Liquid ethane is expected to freeze solutions faster due to faster heat exchange. This faster freezing may have moderated the growth in size but did not prevent it. Sucrose was then added in effort to prevent particle aggregation during cold storage. Like trehalose-containing LNP solutions, particle size tended to increase when sucrose was added (Table 14, entries 4, 6, 8, and 10). However, in contrast to the previous trehalose studies, retention in particle size was observed after thawing LNP solutions containing over 5% sucrose (Table 14, entries 7, 9 and 11).

In another example, Sucrose (5 wt. %) was used as a cryoprotectant, and LNP solutions were diluted with PBS in effort to prevent particle aggregation upon flash freezing. DDAB/DSPC/CHOL/PEG-DMG (50/10/39/1) LNPs were formed via microfluidic mixing with aqueous sodium acetate buffer, and volumetric throughput was held at 2000 μL/min. Particle size was first evaluated in the absence of sucrose (Table 15, entries 1-3). An increase in particle size was observed when particle solutions were frozen in nitrogen slush (Table 15, entry 2) as well as liquid ethane (Table 15, entry 3). A slight increase in particle size was observed after dilution with PBS (Table 15, entries 4-7) as well as with added sucrose (Table 15, entries 8-10). Interestingly, a decrease in particle size was observed upon thawing diluted LNP solution (DF=4) containing sucrose (5 wt. %), leading to particles closely resembling the original sample.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g., a feature, element, characteristic, property, method/process step or limitation) or group of integers (e.g., features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “microfluidic chip” means a device for manipulating nanoliter to microliter volumes of liquid. Such devices frequently contain features such as channels, chambers, and/or valves, and can be fabricated from a variety of different materials, including, but not limited to, glass and polydimethylsiloxane (PDMS). The terms “microfluidic chip” and “microfluidic mixing device,” “droplet microchip chip” and “chip” are used interchangeably.

By “lipid nanoparticle” or “LNP” is meant a particle that comprises a plurality of (i.e., more than one) lipid molecules physically associated with each other by intermolecular forces. The lipid nanoparticles may be, e.g., microspheres (including unilamellar and multiamellar vesicles, e.g., liposomes), a dispersed phase in an emulsion, micelles or an internal phase in a suspension. The lipid nanoparticles have a size of about 1 to about 2,500 nm, about 1 to about 1,500 nm, about 1 to about 1,000 nm, in a sub-embodiment about 50 to about 600 nm, in a sub-embodiment about 50 to about 400 nm, in a sub-embodiment about 50 to about 250 nm, and in a sub-embodiment about 50 to about 150 nm. Unless indicated otherwise, all sizes referred to herein are the average sizes (diameters) of the fully formed nanoparticle, as measured by dynamic light scattering. The nanoparticle sample is diluted in phosphate buffered saline (PBS) so that the count rate is approximately 200-400 kcts. The data is presented as a weighted average of the intensity measure.

The term “nucleic acid” or “nucleic acid molecules” include single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), mRNA (messenger RNA), miRNA (micro-RNA), hpRNA (hairpin RNA), tRNA (transfer RNA), whether charged or discharged with a corresponding acetylated amino acid), and cRNA (complementary RNA). The term “deoxyribonucleic acid” (DNA) is inclusive of cDNA, genomic DNA, and DNA-RNA hybrids. The terms “nucleic acid segment” and “nucleotide sequence segment,” or more generally “segment,” will be understood by those in the art as a functional term that includes both genomic sequences, ribosomal RNA sequences, transfer RNA sequences, messenger RNA sequences, operon sequences, and smaller engineered nucleotide sequences that encoded or may be adapted to encode, peptides, polypeptides, or proteins.

The term “polynucleotide” or “nucleotide” as used herein indicates an organic polymer composed of two or more monomers including nucleotides, nucleosides or analogs thereof. The term “nucleotide” refers to any of several compounds that consist of a ribose or deoxyribose sugar joined to a purine or pyrimidine base and to a phosphate group and that is the basic structural unit of nucleic acids. The term “nucleoside” refers to a compound (such as guanosine or adenosine) that consists of a purine or pyrimidine base combined with deoxyribose or ribose and is found especially in nucleic acids. The term “nucleotide analog” or “nucleoside analog” refers respectively to a nucleotide or nucleoside in which one or more individual atoms have been replaced with a different atom or a with a different functional group. Accordingly, the term “polynucleotide” includes nucleic acids of any length, and in particular DNA, RNA, analogs and fragments thereof. A polynucleotide of three or more nucleotides is also called an “oligomer” or “oligonucleotide.”

The term “messenger ribonucleic acid” (messenger RNA, mRNA) refers to a ribonucleic acid (RNA) molecule that mediates the transfer of genetic information to ribosomes in the cytoplasm, where it serves as a template for protein synthesis. It is synthesized from a DNA template during the process of transcription. A “ribonucleic acid” (RNA) is a polymer of nucleotides linked by a phosphodiester bond, where each nucleotide contains ribose or a modification thereof as the sugar component. Each nucleotide contains an adenine (A), a guanine (G), a cytosine (C), an uracil (U) or a modification thereof as the base. The genetic information in a mRNA molecule is encoded in the sequence of the nucleotide bases of the mRNA molecule, which are arranged into codons consisting of three nucleotide bases each. Each codon encodes for a specific amino acid of the polypeptide, except for the stop codons, which terminate translation (protein synthesis). Within a living cell, mRNA is transported to a ribosome, the site of protein synthesis, where it provides the genetic information for protein synthesis (translation). For a fuller description, see, Alberts B et al. (2007) Molecular Biology of the Cell, Fifth Edition, Garland Science.

In eukaryotes, mRNA is transcribed in vivo at the chromosomes by the cellular enzyme RNA polymerase. During or after transcription in vivo, a 5′ cap (also termed an RNA cap, an RNA 7-methylguanosine cap, or an RNA m7G cap) is added in vivo to the 5′ end of the mRNA. The 5′ cap is terminal 7-methylguanosine residue that is linked through a 5′-5′-triphosphate bond to the first transcribed nucleotide. In addition, most eukaryotic mRNA molecules have a polyadenylyl moiety (“poly(A) tail”) at the 3′ end of the mRNA molecule. In vivo, the eukaryotic cell adds the poly(A) tail after transcription, often at a length of about 250 adenosine residues.

Thus, a typical mature eukaryotic mRNA has a structure that begins at the 5′ end with an mRNA cap nucleotide followed by a 5′ untranslated region (5′UTR) of nucleotides, then an open reading frame that begins with a start codon which is an AUG triplet of nucleotide bases, that is the coding sequence for a protein, and that ends with a stop codon that may be a UAA, UAG, or UGA triplet of nucleotide bases, then a 3′ untranslated region (3′UTR) of nucleotides and ending with a poly-adenosine tail. While the features of the typical mature eukaryotic mRNA are made naturally in a eukaryotic cell in vivo, the same or structurally and functionally equivalent features can be made in vitro using the methods of molecular biology. Accordingly, any RNA having the structure similar to a typical mature eukaryotic mRNA can function as a mRNA and is within the scope of the term “messenger ribonucleic acid”. The mRNA molecule is generally of a size that it can be encapsulated in a lipid nanoparticle of the invention. While the size of a mRNA molecule varies in nature depending upon the identity of the mRNA species that encodes for a particular protein, an average size for a mRNA molecule is average mRNA size is 500-10,000 bases.

When referring to laminar flow, it is generally understood to refer to the flow conditions that fall under the Stokes regime (˜1<Re<˜1000). Re is the Reynolds number defined as Re=pUH/μ, where ρ, U and μ, are the fluid density, the average velocity and dynamic viscosity respectively and H is the characteristic channel dimension.

As used herein, the term “lipid” refers to a group of organic compounds that are esters of fatty acids and are characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

By the term “tunable” as used herein, it is meant that by varying the conditions of the microfluidic mixing chip design, as well as inputs and flow rates among other parameters.

A “channel,” as used herein, means a feature on or in an article (substrate) that at least partially directs the flow of a fluid. The channel can have any cross-sectional shape (circular, oval, triangular, irregular, square or rectangular, or the like) and can be covered or uncovered. In embodiments where it is completely covered, at least one portion of the channel can have a cross-section that is completely enclosed, or the entire channel may be completely enclosed along its entire length with the exception of its inlet(s) and outlet(s). A channel may also have an aspect ratio (length to average cross sectional dimension) of at least 2:1, more-typically at least 3:1, 5:1, or 10:1 or more. An open channel generally will include characteristics that facilitate control over fluid transport, e.g., structural characteristics (an elongated indentation) and/or physical or chemical characteristics (hydrophobicity vs. hydrophilicity) or other characteristics that can exert a force (e.g., a containing force) on a fluid. The fluid within the channel may partially or completely fill the channel. In some cases where an open channel is used, the fluid may be held within the channel, for example, using surface tension (i.e., a concave or convex meniscus).

The channel may be of any size, for example, having a largest dimension perpendicular to fluid flow of less than about 5 nun or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. In some cases, the dimensions of the channel may be chosen such that fluid is able to freely flow through the article or substrate. The dimensions of the channel may also be chosen, for example, to allow a certain volumetric or linear flowrate of fluid in the channel. Of course, the number of channels and the shape of the channels can be varied by any method known to those of ordinary skill in the art. In some cases, more than one channel or capillary may be used. For example, two or more channels may be used, where they are positioned inside each other, positioned adjacent to each other, positioned to intersect with each other, etc.

As used herein, “integral” means that portions of components are joined in such a way that they cannot be separated from each other without cutting or breaking the components from each other.

TABLE 1
Overview of hydrodynamic z-average diameters (D_{z-avg.}) of LNPs
prepared as a function of throughput and lipid concentration.

| | Q_{c} | Q_{EtOH} | Q_{aq.} | EtOH | D_{z-avg.} |
|---|---|---|---|---|---|
| Entry | (μL/min) | (μL/min) | (μL/min) | vol. % | (nm) |
| 1 | 298 | 75 | 112, 112 | 25 | 221 ± 38.7 |
| 2 | 600 | 150 | 225, 225 | 25 | 225 ± 30.2 |
| 3 | 1000 | 250 | 375, 375 | 25 | 232 ± 60 |
| 4 | 2000 | 500 | 750, 750 | 25 | 185 ± 32 |
| 5 | 300 | 30 | 135, 135 | 10 | 397 ± 26.5 |
| 6 | 300 | 100 | 100, 100 | 33 | 226 ± 55 |

TABLE 2
Particle size of diluted LNPs after reconstitution.

| Entry | Dilution Factor^{a} | D_{z-avg.} (nm) |
|---|---|---|
| 1 | 2 | 246 |
| 2 | 3.5 | 210 |
| 3 | 4 | 184 |
| 4^{b} | 5 | 174; 1.3 |
| 5^{b} | 6 | 176; 1.3 |
| 5^{b} | 11 | 144; 1.3 |

^{a}Dilution factor = V_{final}/V_{init.}
^{b}Multimodal size distribution observed.

### Tables

TABLE 3
Summary of particle size and encapsulation efficiency
(EE) for LNPs prepared at higher throughput.

| | Qₜₒₜₐₗ | Q_{EtOH} | Q_{aq.} | V_{EtOH}/ | D_{z-avg.} | EE |
|---|---|---|---|---|---|---|
| entry | (μL/min) | (μL/min) | (μL/min) | V_{aq.} | (nm) | (%) |
| 1 | 299 | 75 | 224 | 1:3 | 138 | 20 |
| 2 | 2000 | 500 | 1500 | 1:3 | 126 | 22 |
| 3 | 2000 | 200 | 1800 | 1:9 | 131 | — |

TABLE 4
Particle sizes obtained as a function of meander
channel distance from droplet junction.

| entry | microchip | D_{z-avg.} (nm) |
|---|---|---|
| 1 | A | 172 |
| 2 | B | 196 |
| 3 | C | 181 |

TABLE 5
Particle sizes obtained as a function
of microchip and lipid composition.

| | | DDAB/DSPC/CHOL/ | | |
|---|---|---|---|---|
| entry | microchip | PEG-DMG | D_{z-avg.} (nm) | PDI |
| 1 | A | 50/10/40/0 | 175 | 0.163 |
| 2 | B | 50/10/40/0 | 133 | 0.270 |
| 3 | A | 50/10/30/10 | 106 | 0.470 |
| 4 | B | 50/10/30/10 | 110 | 0.393 |

TABLE 6
Particle sizes obtained as a function of PEG-DMG content.

| | DDAB/DSPC/CHOL/ | | |
|---|---|---|---|
| entry | PEG-DMG | D_{z-avg.} (nm) | PDI |
| 1 | 50/10/30/10 | 110 | 0.393 |
| 2 | 50/10/35/5 | 106 | 0.471 |
| 3 | 50/10/37.5/2.5 | 106 | 0.466 |
| 4 | 50/10/39/1 | 110 | 0.414 |

TABLE 7
Particle sizes obtained as a function of PEG-
DMG content and volumetric throughput.

| | DDAB/DSPC/CHOL/ | | | |
|---|---|---|---|---|
| entry | PEG-DMG | Qₜₒₜₐₗ (μl/min) | D_{z-avg.} (nm) | PDI |
| 1 | 50/10/30/10 | 2000 | 110 | 0.393 |
| 2 | 50/10/35/5 | 2000 | 106 | 0.471 |
| 3 | 50/10/37.5/2.5 | 2000 | 106 | 0.466 |
| 4 | 50/10/39/1 | 2000 | 110 | 0.414 |
| 5 | 50/10/39/5/0.5 | 2000 | 117 | 0.381 |
| 6 | 50/10/40/0 | 2000 | 133 | 0.270 |
| 7 | 50/10/39/1 | 4000 | 95 | 0.437 |

TABLE 8
Summary of microchannel dimensions after thermal annealing.

| Channel | Channel | Channel | Cross-sectional |
|---|---|---|---|
| Segment | Depth (μm) | Width (μm) | Area (μm²) |
| Inlet | 360 | 1085 | 3.9 × 10⁵ |
| Meander | 261 | 1221 | 3.2 × 10⁵ |

TABLE 9
Particle sizes obtained as a function of volumetric throughput.

| | DDAB/DSPC/CHOL/ | | | |
|---|---|---|---|---|
| entry | PEG-DMG | Qₜₒₜₐₗ (μl/min) | D_{z-avg.} (nm) | PDI |
| 1 | 50/10/39/1 | 2000 | 110 | 0.414 |
| 2 | 50/10/39/1 | 4000 | 95 | 0.437 |
| 3 | 50/10/39/1 | 6000 | 94 | 0.418 |
| 4 | 50/10/39/1 | 8000 | 82 | 0.464 |

TABLE 10
Particle sizes obtained as a function of chip design.

| entry | DDAB/DSPC/CHOL | Microchip | D_{z-avg.} (nm) | PDI |
|---|---|---|---|---|
| 1 | 50/10/40 | Droplet | 202 | 0.188 |
| 2 | 50/10/40 | Hairpin | 165 | 0.194 |
| 3 | 50/10/40 | Meander | 133 | 0.270 |

TABLE 21
Particle sizes obtained as a function of chip design.

| Entry | Microchip | dₘᵢₓ^{a} (mm) | d_{HH}^{b} (mm) | D_{z-avg.} (nm) | PDI |
|---|---|---|---|---|---|
| 1 | A | 137 | 0.664 | 205 | 0.172 |
| 2 | B | 237 | 10.6 | 166 | 0.237 |
| 3 | C | 394 | 26.3 | 165 | 0.194 |
| 4 | D | 636 | 50.5^{c} | 194 | 0.175 |

^{a}mix is the mixing channel length.
^{b}d_{HH} is the distance between hairpin turns.
^{c}average value

TABLE 32
Particle sizes obtained as a function of chip design.

| Entry | Microchip | dₘᵢₓ^{a} (mm) | dᵢⱼ^{b} (mm) | D_{z-avg.} (nm) | PDI |
|---|---|---|---|---|---|
| 1 | A | 137 | 41.8 | 205 | 0.172 |
| 2 | B | 115 | 30.9 | 201 | 0.168 |
| 3 | C | 93.6 | 19.9 | 159 | 0.229 |
| 4 | D | 75.6 | 10.9 | 208 | 0.141 |
| 5 | E | 57.4 | 1.99 | 152 | 0.243 |

^{a}dₘᵢₓ is the mixing channel length.
^{b}d_{HH} is the distance between hairpin turns and the inlet junction.

TABLE 43
Particle sizes obtained as a function of
trehalose content and storage conditions.

| | Trehalose | | | |
|---|---|---|---|---|
| Entry | (wt. %) | T_{storage} (° C.) | D_{z-avg.} (nm) | PDI |
| 1 | 0 | 22 | 125 | 0.367 |
| 2 | 0 | −20 | 309 | 0.261 |
| 3 | 0 | −80 | 184 | 0.156 |
| 4 | 20 | 22 | 307 | 0.168 |
| 5^{a} | 20 | 22 | 293 | 0.175 |
| 6 | 20 | −20 | 296 | 0.142 |
| 7 | 20 | −80 | 297 | 0.142 |
| 8 | 5 | 22 | 156 | 0.182 |

^{a}trehalose incorporated during microfluidic mixing.

TABLE 54
Particle sizes obtained as a function of
sucrose content and storage conditions.

| | Sucrose | | | | |
|---|---|---|---|---|---|
| Entry | (wt. %) | T_{storage} (° C.) | | D_{z-avg.} (nm) | PDI |
| 1 | 0 | 22 | | 111 | 0.378 |
| 2 | 0 | −196 | (LN₂) | 151 | 0.167 |
| 3 | 0 | −188 | (LEt) | 139 | 0.201 |
| 4 | 1 | 22 | | 147 | 0.190 |
| 5 | 1 | −196 | (LN₂) | 167 | 0.046 |
| 6 | 5 | 22 | | 173 | 0.141 |
| 7 | 5 | −196 | (LN₂) | 174 | 0.070 |
| 8 | 10 | 22 | | 182 | 0.188 |
| 9 | 10 | −196 | (LN₂) | 196 | 0.098 |
| 10 | 20 | 22 | | 234 | 0.197 |
| 11 | 20 | −196 | (LN₂) | 233 | 0.200 |

TABLE 65
Particle sizes obtained as a function of sucrose
content, dilution, and storage conditions.

| | Sucrose | Dilution | | | | |
|---|---|---|---|---|---|---|
| Entry | (wt. %) | Factor (DF) | T_{storage} (° C.) | | D_{z-avg.} (nm) | PDI |
| 1 | 0 | n/a | 22 | | 111 | 0.378 |
| 2 | 0 | n/a | −196 | (N2 slush) | 157 | 0.118 |
| 3 | 0 | n/a | −188 | (LEt) | 139 | 0.201 |
| 4 | 0 | 2 | 22 | | 128 | 0.202 |
| 5 | 0 | 2 | −188 | (LEt) | 144 | 0.006 |
| 6 | 0 | 4 | 22 | | 152 | 0.160 |
| 7 | 0 | 4 | −188 | (LEt) | 156 | 0.075 |
| 8 | 5 | 2 | 22 | | 164 | 0.110 |
| 9 | 5 | 2 | −188 | (LEt) | 160 | 0.141 |
| 10 | 5 | 4 | 22 | | 192 | 0.248 |
| 11 | 5 | 4 | −188 | (LEt) | 118 | 0.184 |

### REFERENCES

1. Belliveau, N. M.; Huft, J.; Lin, P. J.; Chen, S.; Leung, A. K.; Leaver, T. J.; Wild, A. W.; Lee, J. B.; Taylor, R. J.; Tam, Y. K.; Hansen, C. L.; Cullis, P. R., Microfluidic Synthesis of Highly Potent Limit-size Lipid Nanoparticles for In Vivo Delivery of siRNA. Mol. Ther. Nucleic Acids 2012, 1, e37.
2. Crawford, R.; Dogdas, B.; Keough, E.; Haas, R. M.; Wepukhulu, W.; Krotzer, S.; Burke, P. A.; Sepp-Lorenzino, L.; Bagchi, A.; Howell, B. J., Analysis of lipid nanoparticles by Cryo-EM for characterizing siRNA delivery vehicles. Int. J. Pharm. 2011, 403, 237-244.
3. Dolomite Microfluidics Microfluidic Calculator. https://www.dolomite-microfluidics.com/support/microfluidic-calculator (accessed Oct. 15, 2020).

What is claimed is:

1. A microfluidic chip comprising:
at least one lipid insertion channel configured to direct a flow of a lipid solution containing a quantity of oligonucleotides;
one or more buffer channels configured to direct a flow of a buffer solution;
an inlet junction positioned at the intersection of the lipid insertion and buffer channels;
a mixing channel in fluid communication with said inlet junction and configured to direct a flow of a lipid and buffer solution to an outlet channel, wherein said mixing channel includes one or more of:
a meander channel in fluid communication with said inlet junction and configured to direct a flow of the lipid and buffer solution to an outlet channel;
one or more hairpin turns in fluid communication with said inlet junction and configured to direct a flow of the lipid and buffer solution to an outlet channel; and
wherein said lipid solution, as it is passes through said mixing channel, forms a plurality of lipid nano-particles (LNPs) encapsulating the oligonucleotides; and
a receiving container configured to collect said LNPs.

2. The microfluidic chip of claim 1, wherein said lipid solution comprises at least one cationic lipid, at least one neutral lipid, at least one sterol, and at least one co-surfactant.

3. The microfluidic chip of claim 2, wherein said lipid solution comprises:
a cationic lipid comprising dimethyldioctadecylammonium bromide (DDAB),
a neutral lipid comprising 1,2-distearoyl-sn-glycero-3-phosphocholine (DSPC),
a sterol/lipid comprising cholesterol (CHOL), and
a cosurfactant comprising 1,2-dimyristoyl-rac-glycero-3-methoxypolyethylene glycol (PEG-DMG).

4. The microfluidic chip of claim 3, wherein the components of the lipid solution have an approximate molar ratio of:
DDAB=50%;
DSPC=10%;
CHOL=39%; and
PEG-DMG=1%.

5. The microfluidic chip of any of claims 1-4, wherein the quantity of oligonucleotides comprise a quantity of RNA oligonucleotides.

6. The microfluidic chip of claim 1, wherein the flow of fluid through the channels of said microfluidic chip comprises a volumetric throughput (Qₜₒₜₐₗ) between 1000 μL/min and 8000 μL/min.

7. The microfluidic chip of claim 1, wherein said meander channel comprises a plurality of meander channels.

8. The microfluidic chip of claim 7, wherein said plurality of meander channels comprises at least one meander segment.

9. The microfluidic chip of claim 1, wherein said one or more hairpin turns comprising a plurality of hairpin turns.

10. The microfluidic chip of claims 1, and 7-9, wherein the onset length between said inlet junction and said first meander channel and/or said first hairpin turn is variable.

11. The microfluidic chip of claims 1, and 7-10, wherein said meander channels and/or hairpin turns include a variable mixing channel length (dₘᵢₓ).

12. The microfluidic chip of claims 1, and 7-10, wherein the distance between the inlet junction and said first meander channel and/or said first hairpin turn (dᵢⱼ) is variable.

13. The microfluidic chip of claims 1, and 7-10, wherein said the distance between said plurality of hairpin turns (dim) is variable.

14. The microfluidic chip of claim 1, and further comprising a cryoprotectant added to said LNPs.

15. The microfluidic chip of claim 14, wherein said cryoprotectant is trehalose or sucrose.

16. The microfluidic chip of any claim above wherein said LNPs have a particle size between 420 nanometers (nm) and 82 nm.

17. The microfluidic chip of any claim above wherein said LNPs have an encapsulation efficiency of at least 71%.

18. The microfluidic chip of any claim above wherein said LNPs are lyophilized.

19. The microfluidic chip of claim 18, wherein the lyophilized LNPs are reconstituted.

20. The microfluidic chip of claim 19, wherein said reconstituted LNPs have at least a 90% retention of encapsulated RNA.

21. A pharmaceutical composition containing a LNP of any claim above.

22. A pharmaceutical composition of claim 21, wherein said pharmaceutical composition is a vaccine.

23. Administering a therapeutically effective amount of a pharmaceutical composition or vaccine of any of claim 21 or 22, to a subject in need thereof.

24. A method of producing lipid nano-particles (LNPs) comprising the steps:
establishing a microfluidic mixing chip having a lipid insertion channel and one or more buffer channels;
directing a lipid solution containing a quantity of oligonucleotides through said lipid insertion channel;
directing a buffer solution through said one or more buffer channels;
mixing said lipid solution containing a quantity of oligonucleotides and said buffer solution at an inlet junction;
directing said solution through a mixing channel having one or more of:
a meander channel in fluid communication with said inlet junction and configured to direct a flow of the lipid and buffer solution to an outlet channel;
one or more hairpin turns in fluid communication with said inlet junction and configured to direct a flow of the lipid and buffer solution to an outlet channel; and
forming a plurality of LNPs encapsulating the oligonucleotides; and
collecting and optionally isolating said LNPs.

25. The method of claim 24, wherein said lipid solution comprises at least one cationic lipid, at least one neutral lipid, at least one sterol, and at least one co-surfactant.

26. The method of claim 25, wherein said lipid solution comprises:
a cationic lipid comprising dimethyldioctadecylammonium bromide (DDAB),
a neutral lipid comprising 1,2-distearoyl-sn-glycero-3-phosphocholine (DSPC),
a sterol/lipid comprising cholesterol (CHOL), and
a cosurfactant comprising 1,2-dimyristoyl-rac-glycero-3-methoxypolyethylene glycol (PEG-DMG).

27. The method of claim 26, wherein the components of the lipid solution have an approximate molar ratio of:
DDAB=50%;
DSPC=10%;
CHOL=39%; and
PEG-DMG=1%.

28. The method of any of claims 24-27, wherein quantity said of oligonucleotides comprise a quantity of RNA oligonucleotides.

29. The method of claim 24, wherein the flow of fluid through the channels of said microfluidic chip comprises a volumetric throughput (Qₜₒₜₐₗ) between 1000 μL/min and 8000 μL/min.

30. The method of claim 24, wherein said meander channel comprises a plurality of meander channels.

31. The method of claim 30, wherein said plurality of meander channels comprises at least one meander segment.

32. The method of claim 24, wherein said one or more hairpin turns comprising a plurality of hairpin turns.

33. The method of claims 24, and 30-32, wherein the onset length between said inlet junction and said first meander channel and/or said first hairpin turn is variable.

34. The method of claims 24, and 30-33, wherein said meander channels and/or hairpin turns include a variable mixing channel length (dₘᵢₓ).

35. The method of claims 24, and 30-33, wherein the distance between the inlet junction and said first meander channel and/or said first hairpin turn (dᵢⱼ) is variable.

36. The method of claims 24, and 30-33, wherein said the distance between said plurality of hairpin turns (d_{HH}) is variable.

37. The method of claim 24, and further comprising the step of adding a cryoprotectant added to said LNPs.

38. The method of claim 37, wherein said cryoprotectant is trehalose or sucrose.

39. The method of any claim above wherein said LNPs have a particle size between 420 nanometers (nm) and 82 nm.

40. The method of any claim above wherein said LNPs have an encapsulation efficiency of at least 71%.

41. The method of any claim above and further comprising the step of lyophilizing said LNPs.

42. The method of claim 41, and further comprising the step of reconstituting the lyophilized LNPs.

43. The method of claim 42, wherein said reconstituted LNPs have at least a 90% retention of encapsulated RNA.

44. A pharmaceutical compositions containing a LNP produced by the method of any claim above.

45. A pharmaceutical composition of claim 44, wherein said pharmaceutical composition is a vaccine.

46. Administering a therapeutically effective amount of a pharmaceutical composition or vaccine of any of claim 44 or 45, to a subject in need thereof.

47. A microfluidic chip for the production of lipid nano-particles (LNPs) having one or more meander channels and/or hairpin turns.

48. A lipid solution for the production of lipid nano-particles (LNPs) comprising:
a cationic lipid comprising dimethyldioctadecylammonium bromide (DDAB),
a neutral lipid comprising 1,2-distearoyl-sn-glycero-3-phosphocholine (DSPC),
a sterol/lipid comprising cholesterol (CHOL), and
a cosurfactant comprising 1,2-dimyristoyl-rac-glycero-3-methoxypolyethylene glycol (PEG-DMG).

49. The solution of claim 48, wherein said wherein the components of the lipid solution have a molar ratio:
DDAB=50%;
DSPC=10%;
CHOL=39%; and
PEG-DMG=1%.

50. The solution of claim 48, and further comprising a quantity of oligonucleotides, or a quantity of mRNA oligonucleotides.

51. A microfluidic mixing chip for the production of lipid nano-particles (LNPs) having:
at least one lipid insertion channel;
one or more buffer channels;
an inlet junction;
a mixing channel fluid, wherein said mixing channel optionally includes one or more of:
a meander channel; and
one or more hairpin turns.

52. The microfluidic mixing chip of claim 51, wherein said microfluidic mixing chip comprises a microfluidic mixing chip selected from the group consisting of: [chemical structure]
